(19) United States
(12) Patent Application Publication
(10) Pub. No.: US 2025/0352087 A1
(43) Pub. Date: Nov. 20, 2025

(54) Motion Capture and Biomechanical Assessment of Goal-Directed Movements

(71) Applicant: The Regents of the University of California, Oakland, CA (US)

(72) Inventors: Jeannie Bailey, San Francisco, CA (US); Ryan Halvorson, San Francisco, CA (US)

(21) Appl. No.: 18/871,774

(22) PCT Filed: Jul. 6, 2023
(86) PCT No.: PCT/US2023/026998

§ 371 Date: Dec. 4, 2024

Related U.S. Application Data
(60) Provisional application No. 63/358,769, filed on Jul. 6, 2022.

(51) Int. Cl. A61B 5/11; A61B 5/00; G16H 50/30; G16H 50/70

(52) U.S. Cl. CPC A61B 5/112; A61B 5/1116; A61B 5/1128; A61B 5/4842; A61B 5/4848; A61B 5/7275; A61B 5/742; G16H 50/30; G16H 50/70; A61B 2503/08; A61B 2503/10; A61B 2503/20; A61B 2505/07; A61B 2505/09; A61B 2560/0487

(57) ABSTRACT

The inventors discovered that three-dimensional posture and time series motion data are capable of providing robust, accurate and objective assessments of patient musculoskeletal health. Through the coupling of novel kinematic modeling and dimensionality reduction techniques, the invention is able to utilize posture and motion trajectory data in order to identify various neuromuscular and musculoskeletal conditions previously indistinguishable through the use of conventional clinical assessments. Further, by leveraging recent advancements in motion capture technologies, the invention provides approaches and systems adapted for remote implementation, allowing for quantitative and objective assessments to be collected over time and at reduced cost. Methods of generating a biomechanical assessment for a patient are provided.

### CROSS REFERENCE TO APPLICATIONS

This application claims priority to the filing date of U.S. Provisional Application Ser. No. 63/358,769, filed on Jul. 6, 2022, the disclosure of which application is incorporated herein by reference.

### INTRODUCTION

Musculoskeletal conditions commonly impede patient biomechanical function. However, despite the prevalence of such conditions, largely subjective musculoskeletal physical examinations that are limited by poor accuracy, reliability, and repeatability are continued to be relied upon for the assessment of musculoskeletal health. One example of a functional test for the lower extremity is the star excursion balance test (SEBT). The SEBT is an assessment of dynamic postural control during which a subject balances on one leg and maximally reaches in each of eight directions with the contralateral leg without falling or shifting weight to the reaching leg. The SEBT has been validated and utilized in various patient populations to study conditions such as osteoarthritis (OA), patellofemoral pain, ankle instability, ligament reconstructions, lower back pain, and athletic injuries. However, administration of the SEBT is prone to error as all eight scores must be recorded manually, often resulting in poor intra-rater and inter-rater reliabilities.

To address these limitations, others have attempted to validate the administration of the SEBT using motion capture technology. However, these technologies often require high-cost motion capture systems and trained personnel operating in a specialized, pre-calibrated testing environment, with subjects having to wear multiple markers to aid computer vision. These factors have limited the widespread adoption of these technologies in clinical settings and complicated the development of large clinical datasets that are necessary to estimate population and disease specific distributions.

### SUMMARY

The inventors discovered that three-dimensional posture and time series motion data are capable of providing robust, accurate and objective assessments of patient musculoskeletal health. Through the coupling of novel kinematic modeling and dimensionality reduction techniques, the invention is able to utilize posture and motion trajectory data in order to identify various neuromuscular and musculoskeletal conditions previously indistinguishable through the use of conventional clinical assessments. Further, by leveraging recent advancements in motion capture technologies, the invention provides approaches and systems adapted for remote implementation, allowing for quantitative and objective assessments to be collected over time and at reduced cost. Thus, the methods and systems of the invention, e.g., as described in greater detail below allow for more informed clinical decision-making leading to improved patient outcomes.

Methods of generating a biomechanical assessment for a patient are provided. Aspects of the methods include: obtaining a visual recording of the subject performing one or more goal-directed movements; extracting three-dimensional time series data from the visual recording for a plurality of body landmarks of the subject; processing the time series data; generating one or more biomedical outcome metrics from the processed time series data; and producing the biomechanical assessment for the subject from the one or more biomedical outcome metrics. Also provided are systems for use in practicing the methods of the invention.

### BRIEF DESCRIPTION OF THE FIGURES

FIGS. 1A to 1B depict SEBT reach directions and provide a visual overview of Generalized Procrustes Analysis (GPA). (A) an illustration of SEBT configuration, floor grid, and camera orientation. (B) a visual overview of GPA with Procrustes superimposition.

FIG. 2 provides a table illustrating the variance explained by each principal component for each reach direction between multiple subjects for an experiment performed in accordance with an embodiment of invention.

FIGS. 3A to 3B provide the results of principal component analysis of postures at maximal reach for an experiment performed in accordance with an embodiment of invention. (A) the posture of each subject at maximum reach is plotted in principal component space along PC1 and PC2 (top) as well as PC1 and PC3 (bottom). (B) provides histograms depicting raw principal component values for each subject grouped according to cohort.

FIGS. 4A to 4B illustrate reach trajectories by disease state. (A) reach trajectories are displayed in principal component space for each of the eight reach directions. (B) every third posture along the mean trajectory for the healthy controls (black) and symptomatic osteoarthritis (red) cohorts are plotted in three-dimensional space along the time axis for the second reach direction.

FIGS. 5A to 5C demonstrate the computation of a Kinematic Deviation Index (KDI) and the correlation of computed KDI metrics with patient reported health measures for an experiment performed in accordance with an embodiment of invention. (A) observed versus ideal trajectories for a representative single subject during a single reach used to compute a KDI metric in accordance with an embodiment of invention. (B) KDI for each subject of the experiment plotted by disease state. (C) correlation of KDI with patient reported health measures, hip disability and osteoarthritis outcome score (HOOS) and knee injury and osteoarthritis outcome score (KOOS).

### DETAILED DESCRIPTION

Methods of generating a biomechanical assessment for a patient are provided. Aspects of the methods include: obtaining a visual recording of the subject performing one or more goal-directed movements; extracting three-dimensional time series data from the visual recording for a plurality of body landmarks of the subject; processing the time series data; generating one or more biomedical outcome metrics from the processed time series data; and producing the biomechanical assessment for the subject from the one or more biomedical outcome metrics. Also provided are systems for use in practicing the methods of the invention.

Before the present invention is described in greater detail, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges and are also encompassed within the invention, subject to any specifically excluded limit in the stated range. Where the stated range includes one or both of the limits, ranges excluding either or both of those included limits are also included in the invention.

Certain ranges are presented herein with numerical values being preceded by the term “about.” The term “about” is used herein to provide literal support for the exact number that it precedes, as well as a number that is near to or approximately the number that the term precedes. In determining whether a number is near to or approximately a specifically recited number, the near or approximating unrecited number may be a number which, in the context in which it is presented, provides the substantial equivalent of the specifically recited number.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, representative illustrative methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present invention is not entitled to antedate such publication by virtue of prior invention. Further, the dates of publication provided may be different from the actual publication dates, which may need to be independently confirmed.

It is noted that, as used herein and in the appended claims, the singular forms “a”, “an”, and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

As will be apparent to those of skill in the art upon reading this disclosure, each of the individual embodiments described and illustrated herein has discrete components and features which may be readily separated from or combined with the features of any of the other several embodiments without departing from the scope or spirit of the present invention. Any recited method can be carried out in the order of events recited or in any other order which is logically possible.

While the apparatus and method has or will be described for the sake of grammatical fluidity with functional explanations, it is to be expressly understood that the claims, unless expressly formulated under 35 U.S.C. § 112, are not to be construed as necessarily limited in any way by the construction of “means” or “steps” limitations, but are to be accorded the full scope of the meaning and equivalents of the definition provided by the claims under the judicial doctrine of equivalents, and in the case where the claims are expressly formulated under 35 U.S.C. § 112 are to be accorded full statutory equivalents under 35 U.S.C. § 112.

### Methods

As summarized above, methods of generating a biomechanical assessment for a patient are provided. Aspects of the methods include: obtaining a visual recording of the subject performing one or more goal-directed movements; extracting three-dimensional time series data from the visual recording for a plurality of body landmarks of the subject; processing the time series data; generating one or more biomedical outcome metrics from the processed time series data; and producing the biomechanical assessment for the subject from the one or more biomedical outcome metrics. Also provided are systems for use in practicing the methods of the invention.

### Goal-Directed Movements

As described above, embodiments of the methods include obtaining a visual recording of a subject performing one or more goal-directed movements. In some embodiments, the goal-directed movement(s) is performed by the subject in order to complete a task. The task may include, but is not limited to, any test or exercise routinely employed by medical professionals to assess or quantify mobility, balance, strength, stability, proprioception, or postural control in a subject (such as, e.g., the Star Excursion Balance Test (SEBT)), athletic exercises (e.g., weightlifting movements), or a task resembling or identical to any task normally performed during the course of the subject's daily life (such as, e.g., a task associated with the employment or a hobby of the subject). In some cases, the goal-directed movement(s) is selected based on a condition experienced by the subject or a circumstance of the subject's daily life.

The terms “subject”, “individual”, “patient”, and “participant” are used interchangeably herein and refer to a subject for which a biomechanical assessment is generated according to the systems and methods disclosed herein. The subject is preferably human, e.g., a child, an adolescent, or an adult (such as a young, middle-aged, or elderly adult) human. In some cases, the subject is sixty years of age or older. In other cases, the subject is younger than sixty years of age. In some instances, the subject has, or is at risk of developing, a mobility disorder. The term mobility disorder is used herein to refer to a group of conditions that affect the ability of a subject to move one or more of their body parts at a normal functional capacity (e.g., freely and without pain). The mobility disorder may include neuromuscular movement disorders and/or musculoskeletal (MSK) movement disorders. Neuromuscular movement disorders may include, but are not limited to, amyotrophic lateral sclerosis (ALS), Charcot-Marie-Tooth disease, multiple sclerosis (MS), muscular dystrophy, myasthenia gravis, myopathy, myositis, peripheral neuropathy, etc. MSK movement disorders may include, but are not limited to, arthritis (such as, e.g., osteoarthritis), tendonitis, a tendon or myotendinous tear, a hernia, chronic health problems such as, e.g., chronic pain or chronic problems associated with bad posture, etc.

In some cases, the MSK movement disorder may affect the subject's lower back. In some instances, the MSK movement disorder may affect one or both of the subject's knees (such as, e.g., one or both of the subject's menisci, anterior cruciate ligaments (ACLs), or patellar tendons). In some cases, the subject may have experienced an injury such as, e.g., an injury resulting in an MSK movement disorder. In these instances, the injury may be an injury to the subject's back or knees, a muscle strain or a muscle tear, or a sprain. The injury may have occurred at any point in time such as, e.g., longer than a year in the past or more recently than a year in the past. In some cases, the subject has received surgery such as, e.g., orthopedic surgery. The surgery may have occurred at any point in time such as, e.g., longer than a year in the past or more recently than a year in the past. In some instances, the subject's employment or a hobby of the subject may put the subject at an elevated risk of developing an MSK disorder. In some cases, the subject may regularly perform physical training exercises such as, e.g., strength, flexibility, or endurance training exercises. In some instances, the physical training exercises may be performed by the subject during, or for the purpose of, physical therapy. The physical therapy exercises may be performed by the subject in order to, e.g., regain mobility after an injury or surgery as described above, or in order to prevent deterioration of the mobility of one or more body parts resulting from, e.g., an MSK disorder or old age.

As described above, embodiments of the methods include obtaining a visual recording of a subject performing one or more goal-directed movements. By goal-directed movement is meant a movement performed with the intention of achieving a specific predetermined outcome or goal. For example, the goal-directed movement(s) may include any movement where a body part is moved toward a specific location (including moving locations such as, e.g., a ball in motion and/or stationary locations such as, e.g., a marked position on the floor) or where body parts are moved into a specific configuration in relation to each other (e.g., standing up, sitting down, fully extending a limb, etc.). In some embodiments, the goal of the one or more goal-directed movements is easily and readily reproducible for multiple subjects. In other words, all the conditions presented to a subject performing the one or more goal-directed movements that affect the subject's performance of the movement(s) may be easily and readily reproducible. In these instances, the performance of the goal-directed movement across multiple subjects may be quantitatively and/or qualitatively compared without any confounding variables outside of the health and ability of each subject.

The body parts used by the subject to perform the goal-directed movement may include, but are not limited to, the subject's arms, legs, hands, pelvis, hips, back, thorax, neck, ankles, feet, hands, phalanges, or shoulders. In some embodiments, the one or more body parts includes a joint of the subject such as, e.g., a ball and socket joint, saddle joint, hinge joint, condyloid joint, pivot joint, or gliding joint. In embodiments where the one or more body parts includes a ball and socket joint, the ball and socket joint may be one or both of the subject's shoulder or hip joints. In embodiments where the one or more body parts includes a hinge joint, the hinge joint may be one or both of the subject's elbow, knee, or ankle joints or one or more of the subject's interphalangeal joints. In embodiments where the one or more body parts includes a condyloid joint, the condyloid joint may be one or both of the subject's radiocarpal joints. In cases where the one or more body parts includes a joint, the goal-directed movement may include the movement of one or more tendons or muscles associated with the joint. For example, in embodiments where the goal-directed movement includes the movement of the subject's knee joints, the goal-directed movement may further include the movement of one or both of the subject's quadricep tendons, patella tendons, hamstring tendons, or iliotibial bands. The type of movement employed by the one or more body parts of the subject in performing the goal-directed movement may vary and may include, but is not limited to, abduction, adduction, flexion, extension, or circumduction movements.

In some embodiments, the one or more goal-directed movements are performed by the subject in order to complete a task. In some cases, the task may include transitioning the body from a first posture to a second posture (e.g., from sitting to standing). By posture is meant the positioning of the body, or a subset of the body (e.g., the lower body, upper body, back, etc.), as a whole at a given time or for a given purpose. In some embodiments, the task may include any test or exercise routinely employed by medical professionals to assess or quantify mobility, balance, strength, stability, proprioception, or postural control in a subject. In some embodiments, the test or exercise may include, but is not limited to, the Functional Reach Test (FRT), sit to stand tests, the Y Balance Test (YBT), timed “Up and Go” tests, tests included in the Berg Balance Scale (BBS), the Star Excursion Balance Test (SEBT), and any similar variations thereof. In embodiments where the test or exercise is the SEBT, any number of reach directions may be included. In some instances, the SEBT is performed by the subject for all eight reach directions. In other cases, only the reach directions most relevant in ascertaining the presence or severity of a specific mobility disorder, as determined by the analytical methods of the invention described in greater detail below, are performed by the subject.

In some embodiments, the task may include an athletic exercise such as, e.g., a weightlifting exercise (e.g., clean and snatch, weighted front or back squat, deadlift, etc.) or a calisthenic exercise (e.g., jumping, burpees, split squats, walking lunges, etc.). In some embodiments, the task may resemble or be identical to any task normally performed during the course of the subject's daily life. In these instances, the task may include a routinely performed mobility task (such as, e.g., standing, getting into a car, walking up stairs, etc.), a task associated with a hobby of the subject (e.g., a sport or recreational activity such as fishing), or a task associated with the subject's employment. For example, the task may include swinging an object in a particular manner (when, e.g., the subject works as a miner, a construction worker, or a firefighter) or throwing an object in a particular manner (when, e.g., the subject plays baseball, softball, or cricket). In some embodiments, the task may include walking a certain number of steps, for a certain amount of time, or to a certain location. In some embodiments, the goal-directed movement(s) (or, e.g., a specific task completed using goal-directed movements) is selected based on a specific mobility disorder the subject may have or may be at a risk of developing.

In some embodiments, the method further includes providing instructions to the subject guiding the subject through performing the one or more goal-directed movements (or, e.g., the task to be completed by performing the one or more goal-directed movements). For example, instructions may be provided to the subject explaining or conveying how to perform the goal-directed movement(s), when to begin performing the movement(s), when to cease or end performing the movement(s), etc. The instructions may be communicated to the subject through any number of various visual or audio means including, but not limited to, text, audible speech, images, or videos. In some embodiments, the instructions may be communicated to the subject using a display device providing visual information and/or a loudspeaker. The display device may be an electronic display device such as, e.g., a liquid crystal display (LCD), an organic light-emitting diode (OLED) display or an active-matrix organic light-emitting diode (AMOLED) display. In some embodiments, the electronic display device is the screen of a smartphone or personal computer. In some embodiments, the electronic display device may include an augmented reality device, such as, e.g., augmented reality headsets, goggles, glasses, or contact lenses. Examples of augmented reality devices include, but are not limited to, the Apple Vision Pro, Oculus Quest, Lenovo Mirage, Microsoft HoloLens, Google Glass, MERGE AR/VR Headset, Magic Leap, etc. In some embodiments, visual information (e.g., one or more images or videos) is provided to the subject instructing the subject on how to perform the one or more goal-directed movements as described above, e.g., on a step-by-step basis.

As described above, embodiments of the methods may include obtaining a visual recording of a subject performing one or more goal-directed movements. The subject may include any human capable of completing the goal-directed movement(s). In some cases, the subject has, or is at risk of developing, a mobility disorder. In some cases, the subject may have experienced an MSK injury or received orthopedic surgery. In these instances, the subject may be undergoing physical therapy in order to regain mobility. The goal-directed movement may include any goal-directed movement capable of being performed by multiple subjects with easily and readily reproducible conditions. In some embodiments, the one or more goal-directed movements are performed by the subject in order to complete a task. The task may include, but is not limited to, any test or exercise routinely employed by medical professionals to assess or quantify mobility, balance, strength, stability, proprioception, or postural control in a subject (e.g., the SEBT), athletic exercises (e.g., weightlifting movements), or a task resembling or identical to any task normally performed during the course of the subject's daily life (e.g., a task associated with the employment or a hobby of the subject). In some embodiments, the goal-directed movement(s) (or, e.g., a specific task completed using goal-directed movements) is selected based on a specific mobility disorder the subject may have or may be at a risk of developing. The visual recording of the subject performing the one or more goal-directed movements (i.e., as described above) may be obtained in any number of ways using any number of devices, as discussed in greater detail below.

### Obtaining the Visual Recording and Extracting Time Series Data

Embodiments of the methods include obtaining a visual recording of the subject performing one or more goal-directed movements. By obtain is meant to make the visual recording accessible or available for the subsequent steps of the methods (e.g., available for three-dimensional time series data extraction). The visual recording may be obtained through any number of means, and from any available source. In some instances, the visual recording may be generated or created using any recording device capable of generating a sequence of visual images over time. In some embodiments, the recording device may include, but is not limited to, digital cameras or camcorders such as, e.g., three-dimensional depth cameras. In some embodiments of the methods, obtaining the visual recording includes generating the visual recording. After the visual recording is obtained, embodiments of the methods include extracting three-dimensional time series data from the visual recording for a plurality of body landmarks of the subject. The body landmarks may include, but are not limited to, any body part or point on the subject's body providing information as to the posture of the subject or the position of the one or more body parts used by the subject to perform the goal-directed movement. The three-dimensional time series data may be extracted using any number of approaches and techniques, as well as combinations thereof, as is known in the art.

As described above, embodiments of the methods include obtaining a visual recording of a subject performing one or more goal-directed movements. The visual recording may include any visual recording of a sufficient quality. By sufficient quality it is meant the visual recording is capable of being used to produce three-dimensional time series data for a plurality of body landmarks of the subject from which accurate and statistically relevant biomedical outcome metrics may be generated as is described in greater detail below. In some embodiments, the visual recording may be obtained by transmitting the recording from, e.g., an electronic device (e.g., a smartphone, a personal computer, or the recording device used to generate the visual recording), external memory (e.g., a flash drive, hard disk, solid state drive, or cloud storage), or a database. Transmitting can include any manner of sending, passing, or conveying the visual recording to a means for performing a subsequent step or steps of the methods (e.g., a processor, computer program or application, lines of computer code, etc.). In some embodiments of the methods, obtaining the visual recording includes generating the visual recording.

In some embodiments, the recording device may include, but is not limited to, digital cameras or camcorders. In some cases, the digital camera or camcorder is configured to generate three-dimensional data and may include, but is not limited to depth cameras and 3D depth cameras such as, e.g., the Microsoft Kinect, Intel RealSense Depth Camera D435, Vuze Plus 3D 360, MYNT EYE 3D Stereo Camera Depth Sensor, etc. In some embodiments, the recording device may be a smartphone camera or a computer camera (e.g., a webcam). For example, the recording device may be an iPhone camera, an Android camera, a personal computer (PC) camera such as, e.g., a tablet computer camera, a laptop camera (e.g., a MacBook or an XPS laptop camera), etc. In some embodiments, the recording device may include one or more cameras of an augmented reality device, such as one or more cameras of augmented reality headsets, goggles, glasses, or contact lenses. Examples of augmented reality devices include, but are not limited to, the Apple Vision Pro, Oculus Quest, Lenovo Mirage, Microsoft HoloLens, Google Glass, MERGE ARVR Headset, Magic Leap, etc.

In embodiments of the methods, the recording device is capable of generating a visual recording of sufficient quality. For example, the recording device may be capable of generating a visual recording having at least a minimum number of frames per second (FPS) or a minimum resolution. The minimum FPS or minimum resolution may vary, e.g., depending on the size of the subject or the goal-directed movement being performed by the subject. In some embodiments, the recording device is capable of producing a video having fifteen FPS or more, such as twenty-nine FPS or more, or thirty FPS or more, or sixty FPS or more, or two hundred forty FPS or more, or five hundred FPS or more, or one thousand FPS or more, or fifteen thousand FPS or more. In some embodiments, the recording device is capable of producing a video having a resolution of 360p or more, such as 720p or more, or 1080p or more, or 2160p or more, or 4000p or more, or 4320p or more, or 8640p or more.

In some embodiments, the visual recording may be generated by placing or setting the recording device on a stable surface. For example, the recording device may be placed on the floor, on a desk or table, on a tripod, on workout equipment at a gym or in a clinic, etc. In some embodiments, the visual recording may be generated while the recording device is held by a human such as, e.g., the subject or an agent of the subject. In embodiments where the recording device is held by the subject, the subject may record themselves performing the goal-directed movement(s) in a reflective surface such as a mirror. In some embodiments, the recording device may include a stabilizer. In some embodiments, the visual recording may be stabilized using, e.g., computer code or a computer program/algorithm after it has been generated using the recording device. The visual recording may be generated in any environment where the subject can perform the goal-directed movement(s) as described above. For example, the recording may be generated at the subject's home, at the subject's place of work, at a clinic or hospital (or, e.g., other medical establishment), outside, in a gym or workout facility, in a sports stadium or complex, during physical therapy (i.e., at any location where physical therapy occurs such as, e.g., a physical therapy center, office, clinic or studio), etc.

As described above, the obtained visual recording is capable of being used to produce three-dimensional time series data for a plurality of body landmarks of the subject. By body landmark is meant a specific point or feature on the subject's body (i.e., a human body) that can be used for identification or tracking. In some cases, the plurality of body landmarks may include, but is not limited to, one or more of the subject's limbs or joints, or the subject's spine or, e.g., a point thereon such as, e.g., points where bones contact the skin. In some embodiments, the plurality of body landmarks may include, but is not limited to, one or more of the subjects hands, wrists, hips, knees, ankles, feet, elbows, shoulders, scapula, neck, chest, or facial features. In some embodiments, the body landmarks are selected depending on the one or more goal-directed movements performed by the subject. In these instances, the plurality of body landmarks may include the landmarks most suited for the identification or tracking of the one or more body parts used by the subject to perform the goal-directed movement and/or the identification or tracking of the subject's posture or, e.g., changes to the subject's posture. For example, in embodiments where the one or more goal-directed movements are performed in order to complete the SEBT, the plurality of body landmarks may include, but are not limited to, both of the subject's shoulders and hips as well as the knee and ankle of the stance or plant leg (i.e., the leg remaining on the ground). In another case, e.g., where the one or more goal-directed movements are performed in order complete sit to stand tests, squats, or jumps, the plurality of body landmarks may include, but are not limited to, both of the subject's shoulders, hips, knees, and ankles. In some embodiments, the plurality of body landmarks forms a shape characterizing a posture of the subject (e.g., characterizing the subject's back, lower body, or overall posture). In other words, a posture shape (i.e., a shape representing the posture of the subject at a specific moment in time) is defined by the plurality of body landmarks, each landmark of the plurality of body landmarks constituting or composing a vertex of the posture shape.

In some embodiments, the obtained visual recording is capable of being used to produce three-dimensional time series data for a plurality of body landmarks of the subject (i.e., the visual recording is of sufficient quality) without the use of a motion tracking marker or sensor. For example, the recording device may be configured to generate three-dimensional data (the recording device may be, e.g., a 3D depth camera) and have a video resolution sufficient for a computer program or application to accurately and reliably identify and track the plurality of body landmarks during performance of the goal-directed movement(s). In some instances, the recording device may emit a laser beam such as, e.g., an infrared (IR) laser beam, a near infrared (NIR) laser beam, or a laser beam of visible light in order to generate the depth coordinates of the three-dimensional data. The laser can be emitted using any capable diode such as, e.g., a vertical-cavity surface-emitting laser (VCSEL) diode. In these instances, the recording device may include radar, sonar, or a Light Detection and Ranging (LiDAR) scanner. For example, the recording device may be a smartphone camera including a LiDAR scanner such as, e.g., the iPhone 15 Pro. In other cases, two or more body landmarks may be used to generate the depth coordinates of the three-dimensional data. For example, the number of pixels between body landmarks having a set distance therebetween such as, e.g., facial features of the subject and the resolution of the recording device may be in used to calculate the depth coordinates of the three-dimensional data.

In some embodiments, the visual recording may include the use of a motion tracking marker or sensor. In these embodiments, the motion tracking marker or sensor may vary and includes, but is not limited to, a wearable device such as a smartwatch (e.g., Apple watches, Garmin watches, or Fitbit® watches). In some embodiments, the wearable device may include motion sensors (e.g., accelerometers, gyroscopes, and magnetometers), electrical sensors (e.g., electrocardiogram sensors), or light sensors (e.g., photoplethysmography (PPG) sensors). The motion tracking marker or sensor may be worn by or affixed to the subject such as, e.g., a body part of subject performing the one or more goal-directed movements as described above. In some embodiments, the motion tracking marker or sensor includes a visual pattern. For example, a smartwatch may be configured to display a striped pattern, or a striped pattern may be printed on paper and affixed (e.g., taped) to the subject. The visual pattern may be used to determine a distance the motion tracking marker or sensor is from the recording device or a distance the motion tracking marker or sensor has traveled between two sequentially generated visual images using, e.g., the resolution of the recording device and the number of pixels between components of the visual pattern.

As described above, embodiments of the methods include extracting three-dimensional time series data from the visual recording for a plurality of body landmarks of the subject. By time series (i.e., time-stamped) data is meant a series of data points indexed in time order. In some embodiments, the three-dimensional time series data includes the location or position of each of the plurality of body landmarks of the subject in three-dimensional space (i.e., the three-dimensional coordinates of each body landmark) at each timepoint (e.g., each video frame) the body landmark appears in the video recording. The three-dimensional time series data may be extracted using any number of approaches and techniques, as well as combinations thereof, as is known in the art. In some embodiments, the three-dimensional time series data is extracted from the visual recording using a computer program or application. In some embodiments, the three-dimensional time series data is extracted from the visual recording using a machine learning model. In these instances, the machine learning model may include an artificial neural network such as, e.g., a recurrent neural network (RNN), convolutional neural network (CNN), or region-convolutional neural network (R-CNN). The machine learning model may include, but is not limited to, any standard machine learning model, as well as combinations thereof, as is known in the art that is capable of identifying the plurality of body landmarks from a visual image. In some embodiments, the machine learning model includes a deep learning model such as, e.g., a ResNet, InceptionNet, VGGNet, GoogLeNet, AlexNet, EfficientNet, or YOLONet neural network. In embodiments where the machine learning model includes a deep learning model (e.g., an artificial neural network) the model may be three or more layers deep, such as five or more layers deep, or ten or more, or twenty or more, or fifty or more, or one hundred or more. The machine learning model may be trained using any relevant data set or, e.g., any data set that includes visual images labeled with one or more relevant body parts. For example, the machine learning model may be trained, at least in part, using DeepLabCut™, DeepPoseKit, LEAP, SLEAP, or Anipose. In some embodiments, a human (e.g., the subject or a technician) may label one or more images or video frames of the visual recording with one or more relevant body parts. The manually labeled images or video frames of the visual recording may then be used to train the machine learning model. In embodiments where a human labels one or more images of the visual recording, the images selected to be labeled may be outliers. In some embodiments, outlier images are images with a minimum Euclidean distance between two successively labeled points (i.e., images where one or more body landmarks jumps a minimum distance between two successive images or video frames). For example, outlier images may include images where a body part jumps twenty or more pixels between two successive images or video frames. In some embodiments, the machine learning model does not require any additional training after initially receiving or processing the visual recording of the subject performing the goal-directed movement as discussed above.

As described above, embodiments of the methods may include obtaining a visual recording of a subject performing one or more goal-directed movements and extracting three-dimensional time series data from the obtained visual recording for a plurality of body landmarks of the subject. The visual recording may include any visual recording of a sufficient quality and may be obtained through any number of means and from any available source. In some embodiments of the methods, obtaining the visual recording includes generating the visual recording. The visual recording may be generated using any recording device capable of or configured to generate three-dimensional data from which accurate and statistically relevant biomedical outcome metrics may be generated. For example, the recording device may include a 3D depth camera, a smartphone camera, and/or a computer camera and may generate depth coordinates using, e.g., a laser or body landmarks and the resolution of the recording device. In some embodiments, the plurality of body landmarks may include the landmarks most suited for the identification or tracking of the one or more body parts used by the subject to perform the goal-directed movement and/or the identification or tracking of the subject's posture (or, e.g., changes to the subject's posture). For example, in embodiments where the one or more goal-directed movements are performed in order to complete the SEBT, the plurality of body landmarks may include both of the subject's shoulders and hips, as well as the knee and ankle of the stance or plant leg. In some instances, the plurality of body landmarks forms a shape characterizing the subject's posture. In some embodiments, the obtained visual recording is generated without the use of a motion tracking marker or sensor. The three-dimensional time series data may be extracted from the obtained visual recording using any number of approaches and techniques, as well as combinations thereof, as is known in the art. In some embodiments, the three-dimensional time series data is extracted from the visual recording using a computer program or application such as, e.g., a machine learning model. In some cases, the three-dimensional time series data includes the location or position of each of the plurality of body landmarks of the subject in three-dimensional space (i.e., the three-dimensional coordinates of each body landmark) at each timepoint, or each frame, the body landmark is present in the video recording. The extracted three-dimensional time series data may then be processed and used to generate one or more biomedical outcome metrics, as discussed in greater detail below.

### Data Processing Techniques and Biomedical Outcome Metrics

As described above, embodiments of the methods include processing the three-dimensional time series data extracted for a plurality of body landmarks as discussed above. In some embodiments, the processing may include cleaning the time series data and/or applying kinematic modeling techniques to the time series data in order to, e.g., transform the three-dimensional coordinates of each of the plurality of body landmarks. After the three-dimensional time series data is processed, embodiments of the methods include generating one or more biomedical outcome metrics for the patient. By biomedical outcome metric is meant a measurable indicator of a state or condition of one or more components of the musculoskeletal system generated from the one or more goal-directed movements as discussed above. Biomedical outcome metrics, in accordance with embodiments of the methods, may vary and include, but are not limited to, those found below.

In some embodiments, the three-dimensional time series data extracted, e.g., as discussed above may be processed in order to clean the data for further analysis. By cleaning the data is meant the data is altered or filtered in order to, e.g., reduce noise, minimize distortion, better capture a subject's posture at the beginning and/or end or maxima and/or minima of a goal-directed movement (the subject's posture during, e.g., maximal reach for the SEBT), smooth motion data (e.g., body landmark or postural motion data), or increase the accuracy or precision of one or more biomedical outcome metrics generated from the extracted time series data. In some embodiments, the extracted time series data (i.e., the raw body landmark position data) may be cleaned using a filter such as, e.g., a signal processing filter. The filter may be a high pass filter, a low pass filter, a band pass filter, or a notch filter. In some embodiments, the filter may be a linear continuous-time filter including, but not limited to, a Butterworth filter, Chebyshev filter, Savitzky-Golay filter, elliptic (Cauer) filter, Bessel filter, Gaussian filter, Optimum “L” (Legendre) filter, or Linkwitz-Riley filter. In embodiments where it is desired to have a flat frequency response in the passband, a Butterworth filter may be used. For example, in some embodiments a 2nd order Butterworth low pass filter may be used in order to clean the time series data before it is used to generate one or more biomedical outcome metrics as discussed in greater detail below. In some embodiments, cleaning may include omitting or excluding (e.g., deleting) data determined to not be necessary for further analysis such as, e.g., data determined to be outliers or body landmark positional data from frames or timepoints wherein other body landmarks necessary to determine the posture of the subject (e.g., posture shape) at the timepoint were not captured in the recording and subsequently extracted.

In some embodiment, the processing of the extracted time series data includes applying kinematic modeling techniques to the time series data. In some instances, the kinematic modeling techniques are applied after the time series data has been cleaned, e.g., as described in above. The kinematic modeling techniques of the claimed invention are employed in order to analyze and, e.g., quantify, the various configurations (i.e., postures) the subject's body experiences as the subject performs the one or more goal-directed movements.

In embodiments where the plurality of body landmarks forms a shape characterizing a posture of the subject (i.e., where each of the plurality of body landmarks constitutes a vertex of a posture shape), the kinematic modeling techniques may include analyzing and/or quantifying the postural motion of the subject (i.e., the trajectory of the subject's posture) as the subject transitions from a first posture to a second posture during performance of the one or more goal-directed movements as described above. In some cases, the first posture and second posture correspond to easily distinguishable or notable moments or segments of the task completed by performing the one or more goal-directed movements. For example, in embodiments where the task completed is a reach direction of the SEBT, the first posture may be the initial posture of the subject at the beginning of the test (e.g., when standing up straight balancing on a single leg) and the second posture may be the posture of the subject at maximal reach. In embodiments where the task completed is a squat, the first posture may be the posture of the subject when at the lowest point of the squat and the second posture may be the posture of the subject when standing up straight after completing the squat.

In some embodiments, the kinematic modeling technique may include selecting and grouping a plurality of body landmarks that, collectively, are able to differentiate between (and, e.g., capture the characteristics of) postures of the subject's body. The posture(s) may be of the subject's overall body or a subset of the subject's body. In embodiments where the posture(s) are of a subset of the subject's body, the subset for which the posture(s) are analyzed (i.e., during performance of the one or more goal-directed movements) may be selected based on a specific mobility disorder the subject may have or may be at a risk of developing. For example, only the body landmarks necessary for differentiating between and characterizing the different postures of the subject's back or spine may be selected, grouped, and analyzed when, e.g., the subject has a herniated disk.

In some embodiments, the kinematic modeling techniques may include performing one or more statistical shape analysis approaches on the extracted posture shapes or, i.e., the shapes formed by the plurality of body landmarks, each body landmark constituting a vertex of the posture shape(s) and having extracted three-dimensional coordinates at each timepoint, as described above. In some embodiments, the statistical shape analysis may be performed on a single extracted posture shape (i.e., the posture shape at a single timepoint). In other embodiments, the statistical shape analysis may be performed on a plurality of extracted posture shapes such as, e.g., all the extracted posture shapes as the subject's posture transitions from a first posture to a second posture while the subject performs the one or more goal-directed movement, or every extracted posture shape at every timepoint.

In some embodiments, the statistical shape analysis includes normalizing and/or standardizing each posture shape such that influences other than the shape of each extracted posture shape are reduced or eliminated. By shape is meant the external form, contours, or outline of a thing/object or, e.g., all the geometrical information that remains when location, scale and rotational effects are filtered out from an object. In some cases, the statistical shape analysis includes normalizing each posture shape for location, scale and/or rotational effects. In these instances, a mean shape or consensus configuration may be determined for a plurality of posture shapes. The plurality of posture shapes may be generated, e.g., from a single individual performing a single goal-directed movement or completing a single task including goal-directed movements, multiple individuals performing a single goal-directed movement or a single task including goal-directed movements, a single individual performing multiple rounds of a goal-directed movement or completing multiple rounds of a task including goal-directed movements, or multiple individuals performing multiple rounds of a goal-directed movement or completing multiple rounds of a task including goal-directed movements. In some embodiments, normalized posture shapes (i.e., the three-dimensional coordinates of each vertex, or body landmark, of each normalized posture shape) may be transformed into a shape space. In some cases, extracted posture shapes may be normalized by performing a transformation of each posture shape into a shape space. By shape space is meant a multidimensional space wherein each point represents a specific shape. In some embodiments, the normalizing and/or the transforming into shape space is performed using a generalized Procrustes analysis (GPA). In these instances, the shape space may be the Procrustes shape space/coordinates.

In some embodiments, statistical shape analysis may include reducing the dimensionality and/or degrees of freedom of each posture shape (i.e., the vertices, or body landmarks, constituting each posture shape). The dimensionality reduction may be performed using any number of a variety of techniques or approaches. For example, dimensionality reduction approaches may include linear methods (e.g., Principal Component Analysis (PCA), Factor Analysis (FA), Linear Discriminant Analysis (LDA)), non-linear methods (e.g., Kernel PCA, t-distributed Stochastic Neighbor Embedding (t-SNE), Multidimensional Scaling (MDS)), and/or feature selection methods (e.g., Backward elimination, Forward elimination, Random forests). In embodiments where GPA is performed on the three-dimensional coordinates, the GPA may reduce the degrees of freedom by seven degrees (i.e., three degrees of freedom lost for three-dimensional translation, one degree of freedom lost for scaling, three degrees of freedom lost for three-dimensional rotation) such that the Procrustes shape space has seven less dimensions than the total degrees of freedom associated with all the body landmarks of a single posture before GPA (e.g., for six body landmarks in three-dimensions the total degrees of freedom or dimensionality of a posture is eighteen). In these embodiments, the dimensionality of the body landmarks of each posture may further be reduced by performing PCA. For example, PCA may be performed following GPA wherein the dimensions associated with principal components (PCs) below a threshold variance are omitted.

In some embodiments, the statistical shape analysis such as, e.g., the normalizing, transforming, and/or dimensionality reduction components of the statistical shape analysis may be performed using machine learning techniques. The machine learning techniques may include supervised, semi-supervised, and/or unsupervised approaches and may include the training of a machine learning model. The machine learning model, in accordance with embodiments of the methods, may vary and may include, but is not limited to, any of the models discussed below or any standard machine learning model, as well as combinations thereof, as is known in the art. In some embodiments, the machine learning model may include, or be configured to employ, a Random Forest (RF) algorithm. In some embodiments, the machine learning model may include, or be configured to employ, a K-nearest neighbors (KNN), logistic regression, linear discriminant analysis (LDA), and/or XGBoost Decision Trees (XGBoost) algorithm.

As described above, after the extracted three-dimensional time series data (e.g., extracted posture shapes including body landmark vertices) is processed, embodiments of the methods include generating one or more biomedical outcome metrics for the subject. By biomedical outcome metric is meant a measurable indicator of a state or condition of one or more components of the musculoskeletal system generated from the one or more goal-directed movements as discussed above.

In some embodiments, the methods of the present disclosure (e.g., as described above) are performed for a plurality of subjects with known musculoskeletal system states or conditions in order to generate posture shapes as each subject performs the same goal-directed movement(s). The known musculoskeletal system states or conditions may include, but is not limited to, any of the mobility disorders as described above. In some cases, the known musculoskeletal system state or condition may include the severity of one or more of the mobility disorders as described above.

In some cases, the plurality of subjects includes subjects known to have a musculoskeletal system state or condition and subjects known not have the musculoskeletal system state or condition. In these instances, the number of subjects known to have the state or condition and the number of subjects known to be free of the state or condition is sufficient to generate an accurate biomedical outcome metric indicative of the musculoskeletal system state or condition in a subject with unknown status regarding the state or condition. By accurately generate is meant the one or more biomedical outcome metrics meets a standard or threshold of statistical relevance (e.g., as determined by a statistical test such as a T-test or an ANOVA test). In some cases, the statistical shape analysis, as described above, is performed for the posture shapes of the plurality of subjects with known musculoskeletal system state or condition status in order to generate a mean posture shape in shape space that may be used to normalize the posture shape(s) of a subject with unknown status regarding the state or condition (and e.g., generate the one or more biomedical outcome metrics). In some instances, the one or more biomedical outcome metrics (e.g., as described in greater detail below) is generated for each of the plurality of subjects with known musculoskeletal system state or condition status such that, e.g., the scores of the one or more biomedical outcome metrics may be correlated with, and used as an indicator of, the state or condition status for a subject with unknown status regarding the state or condition. In some embodiments, processed time series data may be generated for two or more performances of the subject of the one or more goal-directed movements such as, e.g., three or more, or five or more, or ten or more, or fifty or more.

The one or more biomedical outcome metrics may be generated using a single posture shape from each performance of the one or more goal-directed movements (e.g., static posture), or multiple posture shapes from each performance (e.g., dynamic posture). In some embodiments, one or more biomedical outcome metrics may be generated using PCA. In these instances, the PCA may be performed by first projecting each posture from a shape space (such as, e.g., Procrustes curved shape space) into a tangent space. In these instances, the tangent space may be Euclidian tangent space. In some embodiments, one or more biomedical outcome metrics may include the linear combination of the PCs explaining the highest proportion of variance for a single posture shape experienced during performance of the goal-directed movement(s) (using, e.g., the mean posture shape generated by the plurality of individuals as described above) such as, e.g., the first four PCs explaining the highest proportion of variance. For example, in embodiments where the goal-directed movements are performed in order to complete the SEBT, the posture may include the posture at the time of maximal reach for a specific reach direction.

In some embodiments, one or more biomedical outcome metrics may be generated using a characteristic of a subject's posture shape motion or trajectory as the subject transitions from a first posture to a second posture during performance of the one or more goal-directed movements. In these instances, posture motion may be represented as ordered sequences of postures through shape space. In some embodiments, the one or more characteristics of a posture motion or trajectory may include, but is not limited to, path distance (i.e., the total amount of posture change from the first posture shape to the second posture shape, e.g., in shape space), path shape (i.e., how posture changed), and path orientation (i.e., the angle between the first PC's of posture trajectory). In some embodiments, characteristics of a subject's posture motion or trajectory (e.g., the distance, shape, and/or orientation of the trajectory) may be quantified using statistical tests. In some cases, the statistical tests are used to compare the posture trajectory characteristics of different subjects experiencing different musculoskeletal system states and may include, e.g., Mantel tests.

In some embodiments, the one or more biomedical outcome metrics may include a kinematic deviation index (KDI) quantifying the dynamic postural control exhibited during performance of the one or more goal-directed movements performed by the subject. KDI represents the amount to which a posture trajectory deviates from a theoretical ideal trajectory during performance of the one or more goal-directed movements as described above. In some embodiments, KDI is calculated by projecting posture shapes into tangent space from shape space and, e.g., calculating the deviation between a straight line through tangent space from a first posture to a second posture of the subject and the posture trajectory measured from the subject as they transition from the first posture to the second posture through one or more intermediate postures. In some instances, the deviation between the theoretical ideal trajectory and the measured posture trajectory of the subject is quantified by measuring the sum of squares of the distances between the straight line and the intermediate postures normalized by the trajectory length (e.g. the total amount of postural change).

In some embodiments, one or more biomedical outcome metrics may be generated using machine learning techniques. The machine learning techniques may include supervised, semi-supervised, and/or unsupervised approaches and may include the training of a machine learning model. The machine learning model, in accordance with embodiments of the methods, may vary and may include, but is not limited to, any of the models discussed below or above or any standard machine learning model, as well as combinations thereof, as is known in the art. In some embodiments, the machine learning model may include, or be configured to employ, a Random Forest (RF) algorithm. In some embodiments, the machine learning model may include, or be configured to employ, a K-nearest neighbors (KNN), logistic regression, linear discriminant analysis (LDA), and/or XGBoost Decision Trees (XGBoost) algorithm. In some embodiments, the machine learning model may include an artificial neural network (NN). In some embodiments, the machine learning model is a deep learning model. In these cases, the model may be three or more layers deep, such as five or more layers deep, or ten or more, or twelve or more, or thirty or more, or fifty or more, or one hundred or more. In some embodiments, the data of the one or more goal-directed movements may be provided in an image or number/vector format (e.g., as a sequence of normalized posture shapes provided as images or coordinates). In these instances, the machine learning model may be configured as). In these instances, the machine learning model may include, or be based on, a convolutional neural network (CNN), recurrent neural network (RNN), region-convolutional neural network (R-CNN), etc. In some embodiments, the machine learning model is configured to process sequential input data. In these instances, the machine learning model may include, or be based on, a recurrent neural network (RNN) model or a transformer model. In embodiments where the machine learning model includes an RNN, the RNN may include, e.g., long short-term memory (LSTM) architecture, gated recurrent units (GRUs), or attention (i.e., may employ the attention technique or include an attention unit). In some embodiments, the machine learning model may include, or be based on, the architecture of a transformer model.

In some embodiments, a visual recording may be generated (and e.g., time series data may be extracted) at two or more timepoints to generate two or more of the same biomedical outcome metrics for a subject. In some instances a visual recording may be generated at three or more timepoints to generate three or more of the same biomedical outcome metrics, such as four or more, or five or more, or ten or more. The two or more timepoints may be at least 30 seconds apart from each other, such as at least a day apart from each other, or at least a week apart from each other, or at least a month apart from each other, or at least a year apart from each other. In some instances, a first timepoint of the two or more timepoints may occur after an injury of the subject. In other instances, a first timepoint of the two or more timepoints may occur before an injury of the subject in order to, e.g., function as a baseline. In these instances, a subsequent timepoint may occur after an injury of the subject. In some instances, a first timepoint of the two or more timepoints may occur after the subject has received a medical intervention. In other instances, a first timepoint of the two or more timepoints may occur before the subject has received a medical intervention in order to, e.g., function as a baseline. In these instances, a subsequent timepoint may occur after the subject has received a medical intervention. In some embodiments, two or more of the same biomedical outcome metrics generated at different timepoints are used to determine a level of recovery of the subject after an injury or a surgery. In some embodiments, two or more of the same biomedical outcome metrics generated at different timepoints are used to determine a level of effectiveness of a medical intervention (e.g., physical therapy). In some embodiments, two or more of the same biomedical outcome metrics generated at different timepoints are used to determine a decline in the mobility of the subject. A visual recording may be generated (i.e., a timepoint may occur) every set number of minutes, hours, days or months while the subject is receiving a certain medical treatment or working a certain profession.

In some instances, the one or more biomedical outcome metrics and/or the data from which the metrics were generated may be is associated with an identifier of the subject. The identifier of the subject may vary, where examples of identifiers include, but are not limited to alpha/numeric identifiers (e.g., an identification number or a string of letters and/or numbers), codes such as, e.g., QR codes, barcodes, facial recognition metrics, etc. In some embodiments, the identifier may identify the subject through association with identifying information of the subject such as, but not limited to, the subject's full legal name, contact information, home address, social security number, a body landmark of the subject as discussed above such as, e.g., a facial feature, etc. In these embodiments, the association may occur in a database or in a datasheet (e.g., wherein the identifying information may be found by searching for the identifier). In these cases, it may be relatively difficult or impossible to associate the identifying information of the subject with the identifier without access to the database or the datasheet (i.e., the database or datasheet is secured and/or protected). In some instances the one or more biomedical outcome metrics (or, e.g., the data from which the metrics were generated) and/or the associated identifier may be saved via local storage and/or cloud storage and, e.g., may be saved to a database such as a data warehouse.

In some embodiments, correlations and relationships between health outcomes and one or more biomedical outcome metrics may be determined from previously saved biomedical outcome metrics and the data associated therewith (the data from which the biomedical outcome metrics were previously generated) such as, e.g., the biomedical outcome metrics and associated data saved to a data warehouse as discussed above. In some embodiments, correlations and relationships between the diagnosis of a disease or condition and one or more biomedical outcome metrics may be determined from previously saved biomedical outcome metrics and associated data. In some embodiments, correlations and relationships between the fitness of a subject for performing a task and one or more biomedical outcome metrics may be determined from previously saved biomedical outcome metrics and associated data. The previously saved biomedical outcome metrics and associated data may include biomedical outcome metrics and data generated from the subject presently obtaining the one or more biomedical outcome metrics and/or biomedical outcome metrics and data generated from other subjects for which one or more biomedical outcome metrics and associated data were previously obtained.

As discussed above, correlations and relationships between health outcomes, the diagnosis of a disease or condition, or the fitness of a subject for performing a task and one or more biomedical outcome metrics may be determined from previously saved biomedical outcome metrics and associated data such as, e.g., the biomedical outcome metrics and associated data saved to a data warehouse as discussed above. In some embodiments, the correlations and relationships may be determined, at least in part, using linear mixed-effects (LME) models. In some embodiments, the correlations and relationships may be determined, at least in part, using a package including statistical analysis functions such as, e.g., statsmodels. In some embodiments, the relationship or correlation may be determined, at least in part, using a machine learning model, such as, e.g., a machine learning model including a neural network. In some embodiments, the neural network is a deep learning model that is three or more layers deep, such as five or more layers deep, or ten or more, or twenty or more, or fifty or more, or one hundred or more. In embodiments where the machine learning model may include, but is not limited to, a linear and/or logistic regression model, a linear discriminant analysis model, a support vector machine (SVM) model, a random forest model, or an XGBoost model.

### The Biomedical Assessment

Embodiments of the methods include producing a biomechanical assessment for the subject from one or more generated biomedical outcome metrics as described above. The mobility assessment is a qualitative or quantitative determination regarding one or more musculoskeletal health related matters pertaining to the subject. The biomechanical assessment, generated in accordance with embodiments of the methods, may vary and may include, but is not limited to, any of the components found below.

In some instances, the biomechanical assessment includes a qualitative or quantitative determination regarding one or more musculoskeletal health related matters pertaining to the subject relative to a baseline. The baseline may vary, and in some instances includes a cohort average value, such as an average level or value of a given biomedical outcome metric generated from a population or cohort of interest. By population or cohort is meant a group of people banded together or treated as a group, such as the categories of professionals, e.g., fire fighters or professional athletes, a group of people living in a specified locality, a group of people in the same age range, etc. In some instances, the baseline includes a prior value obtained from the subject, e.g., a value obtained from the subject 1 day prior to the generation of the most recent visual recording, or 1 week prior, or 1 month prior, or 6 months prior, or 1 year prior, or 5 years prior, etc. In such instances, the biomedical outcome metrics may indicate a temporal change of the one or more musculoskeletal health related matters pertaining to the subject.

In some embodiments, the biomechanical assessment includes an interpretation of the one or more biomedical outcome metrics. For example, a relationship or correlation between one or more biomedical outcome metrics and a disease or condition such as, e.g., any of the mobility disorders described above, may be determined. The correlation or relationship can be determined by comparing one or more biomedical outcome metrics generated from healthy patients with one or more biomedical outcome metrics generated from patients diagnosed with a disease or condition (e.g., as described above). In some cases, the correlation or relationship may be generated using machine learning model (e.g., a neural network). In these embodiments, the interpretation may include the likelihood that the subject has a disease or condition (e.g., a potential diagnosis). In these instances, the interpretation may include the severity or stage of the disease or condition. In some embodiments, the interpretation may include the likelihood or risk level the subject may have of developing a disease or condition.

In some embodiments, the interpretation may include a general assessment of the subject's MSK health or the health or condition of a specific component or body part of the subject's MSK system. For example, the interpretation may include a general assessment of the subject's knee joint condition (e.g., knee mobility is overall good, somewhat poor, overall poor, etc.). In some embodiments, the interpretation may include a general assessment of a specific movement performed by the subject (e.g., the quality of the movement). In these embodiments, the interpretation may include a determination regarding whether one or more body parts is compensating for another body part or whether one or more body parts is being compensated for. In some embodiments, the interpretation may include a general assessment of a subject's fitness for performing a task (e.g., a movement) or undertaking a duty or responsibility (e.g., associated with the subject's employment). By fitness is meant the ability of the subject to perform, and/or the risks associated with the subject undertaking, a task or tasks associated with the duty or responsibility. For example, the interpretation may include a general assessment regarding the fitness of a sports professional or recreational athlete for returning to practice.

In some embodiments, the biomechanical assessment may include a suggested next course of action. In embodiments where a next course of action is suggested, the suggested course of action may vary. In some instances, the course of action includes obtaining additional tests or consulting with additional medical professionals. For example, the suggested course of action may include consulting a specialist wherein a secondary opinion may be obtained, or additional testing may be recommended or ordered. In some embodiments, the suggested course of action may include a temporary or permanent modification to the subject's responsibilities of employment. For example, the suggested course of action may include a period of time wherein the subject should avoid performing a particular task or movement. In some embodiments, the suggested course of action may include an explanation regarding typical manners in which an individual may develop a higher risk of developing a disease or condition and steps the subject may take to avoid or mitigate the risk. For example, the suggested course of action may include preventative measures, such as, e.g., a recommended exercise routine or recommended braces (e.g., an ankle or knee brace). In some embodiments, the suggested course of action may include a potential treatment regimen or therapy recommendation. By treatment regimen is meant a treatment plan that specifies the quantity, the schedule, and the duration of treatment. For example, the treatment regimen may include a suggested physical therapy, or a suggested lifestyle change (e.g., dietary or exercise routines, etc.).

In some instances, the biomechanical assessment may include an evolution of MSK system condition, a disease or condition severity, or a future injury risk. By evolution is meant a progression of a metric over time such as, e.g., the progression of MSK system condition, a condition or disease severity, or risk of future injury over time. In some cases, the evolution is generated based at least in part on one or more previously obtained biomechanical assessments or biomedical outcome metrics. In some embodiments, the mobility evolution includes an explanation of how the relevant metric has changed over time. For example, the mobility evolution may include a peak, periods of decline or incline, and whether the metric is in a period of incline or decline at the time the present biomechanical assessment was obtained. In some embodiments, the biomechanical assessment may include an assessment of the effectiveness of a previously suggested next course of action (e.g., as described above). For example, the biomechanical assessment may include an assessment of the effectiveness of previously suggested physical therapy or exercise routines. The assessment of effectiveness may be obtained based on whether the mobility evolution indicates the level of a metric is in a period of incline or decline at the time the present biomechanical assessment was obtained.

In some embodiments, the biomechanical assessment may include one or more mobility scores. By mobility score is meant a quantitative evaluation of the subject's overall mobility, the mobility of one or more body parts of the subject's MSK system, a specific movement performed by the subject, or the subject's fitness for performing a task compared with a baseline. The baseline may vary, and in some instances includes the average of data associated with a cohort of interest. In some instances, the baseline includes prior data obtained for the subject. In embodiments where the one or mobility scores includes an evaluation of a specific goal-directed movement performed by the subject, one or more of the scores may indicate whether one or more body parts of the subject is being compensated for or is compensating for another body part. The one or more mobility scores may be a composite of multiple biomedical outcome metrics, e.g., compared with a baseline.

In some instances, the biomechanical assessment may include one or more personalized insights. A personalized insight may vary and includes, but is not limited to, the detection of an anomaly, a classification, the detection of a cluster, or a forecast. In some instances, the personalized insight includes an insight regarding the subject individually. In other instances, the personalized insight includes an insight regarding a group or cohort in which the subject belongs. In embodiments where the insight includes the detection of an anomaly, the insight may include the identification of unusual data. For example, the insight may be that a specific goal-directed movement performed by the subject is abnormal and what is abnormal about the movement (e.g., when compared to a baseline as described above). In embodiments where the insight includes a classification, the insight may include the identification of a group with similar data to the subject and, e.g., assigning and comparing the results and/or data of the subject to the group. For example, the insight may be that the subject has better hip mobility than 70% of people in their age group. In embodiments where the insight includes the detection of a cluster, the insight may include finding groups with similar results. For example, the insight may be that a profession or hobby has the highest rate of MSK injuries or the fastest decline in overall mobility.

As discussed above, the biomechanical assessment may include one or more personalized insights. In some embodiments, the personalized insight may include a forecast. In some embodiments, the forecast may include a predicted future outcome such as, e.g., a health or mobility outcome prediction for the subject. The health or mobility outcome can be predicted, at least in part, using a biomechanical assessment or biomedical outcome metric obtained as discussed above. For example, the predicted health or mobility outcome may be that the subject has a high risk of developing a specific disease or condition (e.g., arthritis, chronic pain, or knew injury). In some instances, the health or mobility outcome can be predicted at least in part using a machine learning model such as, e.g., a machine learning model that uses an artificial neural network. In some instances, the biomechanical assessment is used to determine if a particular injury, surgery, or medical intervention has affected the subject's predicted health or mobility outcomes. In instances where the subject is recorded at two or more timepoints to generate two or more biomechanical assessments and/or biomedical outcome metrics, the two or more biomechanical assessments and/or biomedical outcome metrics may be used to, e.g., determine any changes in the subject's overall mobility, the mobility of one or more body parts of the subject's MSK system, the quality of a specific movement performed by the subject, or the subject's fitness for performing a task. In some cases, some combination of the two or more biomechanical assessments and/or biomedical outcome metrics is used to determine if the subject has experienced a decline in mobility.

In some embodiments, the biomechanical assessment may include notes or explanations aiding the subject, or a person associated with the subject, in interpreting the results of the biomechanical assessment. In some embodiments, the biomechanical assessment may include a background section such as, e.g., a background section explaining the purpose of the biomechanical assessment and the implication of certain results. In some embodiments, the biomechanical assessment may include visual means aiding the subject, or a person associated with the subject, in interpreting the findings of the biomechanical assessment (e.g., figures, charts, images, etc.). The visual means may be a component of, or accompany, any of the components the biomechanical assessment is comprised of such as, e.g., any of the components described above.

In some embodiments, the biomechanical assessment may be obtained or generated, at least in part, using a machine learning model such as, e.g., a machine learning model using a neural network. In embodiments wherein a machine learning model is used, any of the components the biomechanical assessment is comprised of such as, e.g., any of the components described above may be generated or obtained using the machine learning model. For example, in embodiments where the biomechanical assessment includes the detection of an anomaly as described above, the detection may be generated or obtained using a machine learning model.

As discussed above, a biomechanical assessment can be generated for a subject from one or more biomedical outcome metrics. In some instances, the biomechanical assessment is generated in real time. By real time is meant the biomechanical assessment is generated during or immediately following generation of the visual recording. In some instances, the biomechanical assessment is generated in two hours or less. In some cases, the biomechanical assessment is generated in one hour or less, such as thirty minutes or less, or twenty minutes or less, or ten minutes or less, or five minutes or less, or one minute or less following generation of the visual recording.

In some instances, the biomechanical assessment is associated with an identifier of the subject. The biomechanical assessment and/or associated identifier may be saved to a database such as, e.g., a database including a data warehouse. In some instances, the data warehouse is used to determine a relationship between health or mobility outcomes, the diagnosis of a disease or condition, the fitness of a subject for performing a task, and one or more biomedical outcome metrics or biomechanical assessment components as discussed above. The relationship may be determined, at least in part, using a machine learning model such as, e.g., a machine learning model including a neural network. In some instances, the determined relationship may be used to generate a subsequent biomechanical assessment.

In some embodiments, the method further includes suggesting preventative measures based on the biomechanical assessment, such as, e.g., recommended equipment (e.g., braces) to avoid potential declines in mobility. In some embodiments, the method further includes providing a therapy recommendation to the subject based on the biomechanical assessment. While the therapy recommendation may vary, in some instances the therapy recommendation includes recommendations regarding the specifics of administering some existing standard of care for the treatment of a disease or condition. In some instances, the method further includes administering the treatment to the subject.

Embodiments of the methods may further include transmitting the biomechanical assessment, e.g., to a health care practitioner, to the subject, to an agent of the subject, etc. In some instances, the biomechanical assessment is received by a computer or mobile device application, such as a smart phone or computer app. In some cases, the biomechanical assessment is received by mail, electronic mail, fax machine, etc. Aspects of the invention further include methods of obtaining a biomechanical assessment, e.g., by using a system of the invention as discussed in greater detail below; and receiving a biomechanical assessment from the system.

### Systems

Aspects of the present disclosure further include systems, such as computer-controlled systems, for practicing embodiments of the above methods. Aspects of the systems include: a display configured to provide visual information instructing the subject to perform one or more goal-directed movements; a digital recording device configured to generate a visual recording of the subject performing the one or more goal-directed movements; a processor configured to receive the visual recording generated by the camera; and memory operably coupled to the processor wherein the memory includes instructions stored thereon, which when executed by the processor, cause the processor to extract three-dimensional time series data from the visual recording for a plurality of body landmarks of the subject, process the time series data, generate one or more biomedical outcome metrics from the processed time series data, and produce a biomechanical assessment for the subject from the one or more biomedical outcome metrics. The systems allow for a biomechanical assessment to be generated for the subject from a recording of the subject performing one or more goal-directed movements, as discussed above.

In some embodiments, the display device providing visual information instructing the subject to perform one or more goal-directed movements (e.g., according to any of the methods as discussed above) may be an electronic display device such as, e.g., a liquid crystal display (LCD), an organic light-emitting diode (OLED) display or an active-matrix organic light-emitting diode (AMOLED) display. In some embodiments, the electronic display device is the screen of a smartphone or personal computer. In some embodiments, the electronic display device may include an augmented reality device, such as, e.g., augmented reality headsets, goggles, glasses, or contact lenses. The augmented reality device may include, but is not limited to, the Apple Vision Pro, Oculus Quest, Lenovo Mirage, Microsoft HoloLens, Google Glass, MERGE AR/VR Headset, Magic Leap, etc.

In some embodiments, the digital recording device may include any device capable of generating a sequence of visual images over time. In some cases, the digital camera or camcorder is configured to generate three-dimensional data and may include, but is not limited to depth cameras and 3D depth cameras such as, e.g., the Microsoft Kinect, Intel RealSense Depth Camera D435, Vuze Plus 3D 360, MYNT EYE 3D Stereo Camera Depth Sensor, etc. In some embodiments, the recording device may be a smartphone camera or a computer camera (e.g., a webcam). For example, the recording device may be an iPhone camera, an Android camera, a personal computer (PC) camera such as, e.g., a tablet computer camera, a laptop camera (e.g., a MacBook or an XPS laptop camera), etc. In some embodiments, the recording device may include one or more cameras of an augmented reality device, such as one or more cameras of augmented reality headsets, goggles, glasses, or contact lenses. Examples of augmented reality devices include, but are not limited to, the Apple Vision Pro, Oculus Quest, Lenovo Mirage, Microsoft HoloLens, Google Glass, MERGE AR/VR Headset, Magic Leap, etc.

In embodiments of the systems, the recording device is capable of generating a visual recording of sufficient quality. For example, the recording device may be capable of generating a visual recording having at least a minimum number of frames per second (FPS) or a minimum resolution. The minimum FPS or minimum resolution may vary, e.g., depending on the size of the subject or the goal-directed movement(s) being performed by the subject. In some embodiments, the recording device is capable of producing a video having fifteen FPS or more, such as twenty-nine FPS or more, or thirty FPS or more, or sixty FPS or more, or two hundred forty FPS or more, or five hundred FPS or more, or one thousand FPS or more, or fifteen thousand FPS or more. In some embodiments, the recording device is capable of producing a video having a resolution of 360p or more, such as 720p or more, or 1080p or more, or 2160p or more, or 4000p or more, or 4320p or more, or 8640p or more. In some embodiments, the recording device is capable of generating an audio recording (e.g., the recording device includes a microphone). In some cases, the system may further include a widget configured to stabilize the recording device such as, e.g., a tripod. In some embodiments, the recording device includes an audio recording component such as, e.g., a microphone.

In some embodiments, the system may further include a motion tracking marker or sensor configured to be worn or affixed to the subject. In some cases, the motion tracking marker or sensor is configured to be worn or affixed to a body part of the subject performing the goal-directed movement(s), as described above. The motion tracking marker or sensor may vary and includes, but is not limited to, a wearable device such as a smartwatch (e.g., Apple watches, Garmin watches, or Fitbit® watches). In some embodiments, the wearable device may include motion sensors (e.g., accelerometers, gyroscopes, and magnetometers), electrical sensors (e.g., electrocardiogram sensors), or light sensors (e.g., photoplethysmography (PPG) sensors). In some embodiments, the motion tracking marker or sensor is configured to produce a visual pattern or emit an audio frequency. For example, a smartwatch may be configured to display a striped pattern, or the striped pattern may be printed on paper and affixed (e.g., taped) to the subject.

The processor may include memory which, when executed by the processor, causes the processor to determine a distance one or more body landmarks is from the recording device or a distance the one or more body landmarks has traveled between two sequentially generated visual images using, e.g., the resolution of the recording device and the number of pixels between one or more body landmarks. The processor may include memory which, when executed by the processor, causes the processor to determine a velocity the one or more body landmarks is traveling away from or towards the recording device.

In some embodiments, the memory includes instructions stored thereon, which when executed by the processor, further cause the processor to extract three-dimensional time series data from the visual recording for a plurality of body landmarks of the subject. In some embodiments, the processor includes instructions stored thereon, which when executed by the processor, further cause the processor to process the extracted time series data according to any of the methods as discussed above. In some embodiments, the processor includes instructions stored thereon, which when executed by the processor, further cause the processor to generate one or more biomedical outcome metrics from the time series data according to any of the methods as discussed above. In some embodiments, the instructions, when executed by the processor, cause the processor to produce a biomechanical assessment for the subject from the one or more generated biomedical outcome metrics according to any of the methods as discussed above.

In some instances the systems further include one or more computers for complete automation or partial automation of the methods described herein. In some embodiments, systems include a computer having a computer readable storage medium with a computer program stored thereon.

In embodiments, the system includes an input module, a processing module and an output module. The subject systems may include both hardware and software components, where the hardware components may take the form of one or more platforms, e.g., in the form of servers, such that the functional elements, i.e., those elements of the system that carry out specific tasks (such as managing input and output of information, processing information, etc.) of the system may be carried out by the execution of software applications on and across the one or more computer platforms represented of the system.

Systems may include a display and operator input device. Operator input devices may, for example, be a touchscreen, a keyboard, a mouse, or the like. The processing module includes a processor which has access to a memory having instructions stored thereon for performing the steps of the subject methods. The processing module may include an operating system, a graphical user interface (GUI) controller, a system memory, memory storage devices, and input-output controllers, cache memory, a data backup unit, and many other devices. The processor may be a commercially available processor or it may be one of other processors that are or will become available. The processor executes the operating system and the operating system interfaces with firmware and hardware in a well-known manner, and facilitates the processor in coordinating and executing the functions of various computer programs that may be written in a variety of programming languages, such as Java, Perl, C, C++, Python, MATLAB, other high-level or low-level languages, as well as combinations thereof, as is known in the art. The operating system, typically in cooperation with the processor, coordinates and executes functions of the other components of the computer. The operating system also provides scheduling, input-output control, file and data management, memory management, and communication control and related services, all in accordance with known techniques. The processor may be any suitable analog or digital system. In some embodiments, the processor includes analog electronics which provide feedback control, such as for example positive or negative feedback control. In some embodiments, the feedback control is of, e.g., goal-directed movement performance.

The system memory may be any of a variety of known or future memory storage devices. Examples include any commonly available random access memory (RAM), magnetic medium such as a resident hard disk or tape, an optical medium such as a read and write compact disc, flash memory devices, or other memory storage device. The memory storage device may be any of a variety of known or future devices, including a compact disk drive, a tape drive, a removable hard disk drive, or a diskette drive. Such types of memory storage devices typically read from, and/or write to, a program storage medium (not shown) such as, respectively, a compact disk, magnetic tape, removable hard disk, or floppy diskette. Any of these program storage media, or others now in use or that may later be developed, may be considered a computer program product. As will be appreciated, these program storage media typically store a computer software program and/or data. Computer software programs, also called computer control logic, typically are stored in system memory and/or the program storage device used in conjunction with the memory storage device.

In some embodiments, a computer program product is described including a computer usable medium having control logic (computer software program, including program code) stored therein. The control logic, when executed by the processor the computer, causes the processor to perform functions described herein. In other embodiments, some functions are implemented primarily in hardware using, for example, a hardware state machine. Implementation of the hardware state machine so as to perform the functions described herein will be apparent to those skilled in the relevant arts.

Memory may be any suitable device in which the processor can store and retrieve data, such as magnetic, optical, or solid-state storage devices (including magnetic or optical disks or tape or RAM, or any other suitable device, either fixed or portable). The processor may include a general-purpose digital microprocessor suitably programmed from a computer readable medium carrying necessary program code. Programming can be provided remotely to processor through a communication channel, or previously saved in a computer program product such as memory or some other portable or fixed computer readable storage medium using any of those devices in connection with memory. For example, a magnetic or optical disk may carry the programming, and can be read by a disk writer/reader. Systems of the invention also include programming, e.g., in the form of computer program products, algorithms for use in practicing the methods as described above. Programming according to the present invention can be recorded on computer readable media, e.g., any medium that can be read and accessed directly by a computer. Such media include, but are not limited to: magnetic storage media, such as floppy discs, hard disc storage medium, and magnetic tape; optical storage media such as CD-ROM; electrical storage media such as RAM and ROM; portable flash drive; and hybrids of these categories such as magnetic/optical storage media.

The processor may also have access to a communication channel to communicate with a user at a remote location. By remote location is meant the user is not directly in contact with the system and relays input information to an input manager from an external device, such as a computer connected to a Wide Area Network (“WAN”), telephone network, satellite network, or any other suitable communication channel, including a mobile telephone (i.e., smartphone).

In some embodiments, systems according to the present disclosure may be configured to include a communication interface. In some embodiments, the communication interface includes a receiver and/or transmitter for communicating with a network and/or another device. The communication interface can be configured for wired or wireless communication, including, but not limited to, radio frequency (RF) communication (e.g., Radio-Frequency Identification (RFID), Zigbee communication protocols, Z-Wave communication protocols, ANT communication protocols, WiFi, infrared, wireless Universal Serial Bus (USB), Ultra Wide Band (UWB), Bluetooth® communication protocols, and cellular communication, such as code division multiple access (CDMA) or Global System for Mobile communications (GSM).

In one embodiment, the communication interface is configured to include one or more communication ports, e.g., physical ports or interfaces such as a USB port, an RS-232 port, or any other suitable electrical connection port to allow data communication between the subject systems and other external devices such as a computer terminal (for example, at a physician's office or in hospital environment) that is configured for similar complementary data communication.

In one embodiment, the communication interface is configured for infrared communication, Bluetooth® communication, or any other suitable wireless communication protocol to enable the subject systems to communicate with other devices such as computer terminals and/or networks, communication enabled mobile telephones, personal digital assistants, or any other communication devices which the user may use in conjunction.

In one embodiment, the communication interface is configured to provide a connection for data transfer utilizing Internet Protocol (IP) through a cell phone network, Short Message Service (SMS), wireless connection to a personal computer (PC) on a Local Area Network (LAN) which is connected to the internet, or WiFi connection to the internet at a WiFi hotspot.

In one embodiment, the subject systems are configured to wirelessly communicate with a server device via the communication interface, e.g., using a common standard such as 802.11 or Bluetooth® RF protocol, or an IrDA infrared protocol. The server device may be another portable device, such as a smart phone, Personal Digital Assistant (PDA) or notebook computer; or a larger device such as a desktop computer, appliance, etc. In some embodiments, the server device has a display, such as a liquid crystal display (LCD), as well as an input device, such as buttons, a keyboard, mouse or touch-screen.

In some embodiments, the communication interface is configured to automatically or semi-automatically communicate data stored in the subject systems, e.g., in an optional data storage unit, with a network or server device using one or more of the communication protocols and/or mechanisms described above.

Output controllers may include controllers for any of a variety of known display devices for presenting information to a user, whether a human or a machine, whether local or remote. If one of the display devices provides visual information, this information typically may be logically and/or physically organized as an array of picture elements. A graphical user interface (GUI) controller may include any of a variety of known or future software programs for providing graphical input and output interfaces between the system and a user, and for processing user inputs. The functional elements of the computer may communicate with each other via system bus. Some of these communications may be accomplished in alternative embodiments using network or other types of remote communications. The output manager may also provide information generated by the processing module to a user at a remote location, e.g., over the Internet, phone or satellite network, in accordance with known techniques. The presentation of data by the output manager may be implemented in accordance with a variety of known techniques. As some examples, data may include CSV, SQL, HTML or XML documents, email or other files, or data in other forms. The data may include Internet URL addresses so that a user may retrieve additional CSV, SQL, HTML, XML, or other documents or data from remote sources. The one or more platforms present in the subject systems may be any type of known computer platform or a type to be developed in the future, although they typically will be of a class of computer commonly referred to as servers. However, they may also be a main-frame computer, a workstation, or other computer type. They may be connected via any known or future type of cabling or other communication system including wireless systems, either networked or otherwise. They may be co-located or they may be physically separated. Various operating systems may be employed on any of the computer platforms, possibly depending on the type and/or make of computer platform chosen. Appropriate operating systems include Windows, Apple operating systems (e.g., iOS, macOS, watchOS, iPadOS, visionOS), Android, Oracle Solaris, Linux, IBM i, Unix, and others.

Aspects of the present disclosure further include non-transitory computer readable storage mediums having instructions for practicing the subject methods. Computer readable storage mediums may be employed on one or more computers for complete automation or partial automation of a system for practicing methods described herein. In certain embodiments, instructions in accordance with the method described herein can be coded onto a computer-readable medium in the form of “programming”, where the term “computer readable medium” as used herein refers to any non-transitory storage medium that participates in providing instructions and data to a computer for execution and processing. Examples of suitable non-transitory storage media include a floppy disk, hard disk, optical disk, magneto-optical disk, CD-ROM, CD-R, magnetic tape, non-volatile memory card, ROM, DVD-ROM, Blue-ray disk, solid state disk, and network attached storage (NAS), whether or not such devices are internal or external to the computer. A file containing information can be “stored” on computer readable medium, where “storing” means recording information such that it is accessible and retrievable at a later date by a computer. The computer-implemented method described herein can be executed using programming that can be written in one or more of any number of computer programming languages. Such languages include, for example, Python, Java, Java Script, C, C#, C++, Go, R, Swift, PHP, as well as many others.

The non-transitory computer readable storage medium may be employed on one or more computer systems having a display and operator input device. Operator input devices may, for example, be a keyboard, mouse, or the like. The processing module includes a processor which has access to a memory having instructions stored thereon for performing the steps of the subject methods. The processing module may include an operating system, a graphical user interface (GUI) controller, a system memory, memory storage devices, and input-output controllers, cache memory, a data backup unit, and many other devices. The processor may be a commercially available processor or it may be one of other processors that are or will become available. The processor executes the operating system and the operating system interfaces with firmware and hardware in a well-known manner, and facilitates the processor in coordinating and executing the functions of various computer programs that may be written in a variety of programming languages, such as those mentioned above, other high level or low level languages, as well as combinations thereof, as is known in the art. The operating system, typically in cooperation with the processor, coordinates and executes functions of the other components of the computer. The operating system also provides scheduling, input-output control, file and data management, memory management, and communication control and related services, all in accordance with known techniques.

### Utility

The methods and systems of the invention, e.g., as described above, find use in a variety of applications where it is desirable to provide robust, accurate and objective assessments of patient musculoskeletal health. In some embodiments, the methods and systems described herein find use when it is desirable to assess or diagnose MSK pathologies previously indistinguishable through the use of conventional clinical assessments. Embodiments of the present disclosure find use in applications wherein it is desired to acquire additional health and mobility information through non-invasive and remote diagnostic procedures in order to, e.g., facilitate informed clinical decision-making leading to improved patient outcomes. In some embodiments, the subject methods and systems may facilitate a determination regarding the recovery of a subject after an injury or surgery or the effectiveness of a method of treatment (e.g., physical therapy) through the generation of useful data by low or minimally trained technicians or without a technician. In some embodiments, the subject methods and systems may facilitate diagnosis for one or more conditions, insight on one or more health risks, or recommendations for one or more therapies or treatments.

### EXAMPLES OF NON-LIMITING ASPECTS OF THE DISCLOSURE

Aspects, including embodiments, of the present subject matter described above may be beneficial alone or in combination, with one or more other aspects or embodiments. Without limiting the foregoing description, certain non-limiting aspects of the disclosure numbered 1-115 are provided below. As will be apparent to those of skill in the art upon reading this disclosure, each of the individually numbered aspects may be used or combined with any of the preceding or following individually numbered aspects. This is intended to provide support for all such combinations of aspects and is not limited to combinations of aspects explicitly provided below:

1. A method of generating a biomechanical assessment for a subject, the method comprising:
obtaining a visual recording of the subject performing one or more goal-directed movement;
extracting three-dimensional time series data from the visual recording for a plurality of body landmarks of the subject;
processing the time series data;
generating one or more biomedical outcome metrics from the processed time series data; and
producing the biomechanical assessment for the subject from the one or more biomedical outcome metrics.
   2. The method according to Aspect 1, wherein the goal-directed movement comprises a gait movement.
   3. The method according to Aspect 1, wherein the goal-directed movement is performed by the subject in order to complete a task.
   4. The method according to Aspect 3, wherein the task is a functional balance task.
   5. The method according to Aspect 4, wherein the task comprises one or more of the directional reaching tasks of the star excursion balance test (SEBT).
   6. The method according to Aspect 2, wherein the task resembles or is identical to a task associated with the subject's employment.
   7. The method according to Aspect 2, wherein the task is an athletic exercise.
   8. The method according to any of Aspects 3 to 7, wherein the task comprises transitioning the body from a first posture to a second posture.
   9. The method according to any of the preceding aspects, wherein instructions are provided to the subject guiding the subject through performing the one or more goal-directed movements.
   10. The method according to any of the preceding aspects, wherein the visual recording is generated without the use of a motion tracking marker.
   11. The method according to any of the preceding aspects, wherein the visual recording is generated using a three-dimensional depth camera.
   12. The method according to any of the preceding aspects, wherein the visual recording is generated using a webcam or smartphone.
   13. The method according to any of the preceding aspects, wherein the visual recording is generated using an augmented reality device.
   14. The method according to any of the preceding aspects, wherein the visual recording is generated at 29 or more frames per second.
   15. The method according to any of the preceding aspects, wherein the visual recording is generated at a resolution of 360p or more.
   16. The method according to any of the preceding aspects, wherein the visual recording is generated at the subject's home.
   17. The method according to any of Aspects 1 to 16, wherein the visual recording is generated at a clinic or hospital.
   18. The method according to any of Aspects 1 to 16, wherein the visual recording is generated at a physical therapy office or studio.
   19. The method according to any of the preceding aspects, wherein one or more of the plurality of body landmarks comprises a bone or joint of the subject.
   20. The method according to Aspect 19, wherein one or more of the plurality of body landmarks is selected from the group consisting of one or both of the ankles, knees, hips, and shoulders of the subject.
   21. The method according to any of the preceding aspects, wherein one or more of the plurality of body landmarks is a facial feature of the subject.
   22. The method according to any of the preceding aspects, wherein the plurality of body landmarks forms a shape characterizing the subject's posture.
   23. The method according to any of the preceding aspects, wherein the extracted time series data comprises three-dimensional coordinates for the plurality of body landmarks.
   24. The method according to Aspect 23, wherein the processing comprises filtering the extracted time series data.
   25. The method according to Aspect 24, wherein the extracted time series data is filtered using a low pass filter.
   26. The method according to Aspect 25, wherein low pass filter is a Butterworth filter.
   27. The method according to any of Aspects 23 to 26, wherein the time series data is extracted using a machine learning model.
   28. The method according to Aspect 27, wherein the machine learning model comprises a neural network.
   29. The method according to Aspect 28, wherein the neural network is a convolutional neural network.
   30. The method according to any of Aspects 23 to 29, wherein the processing comprises applying kinematic modeling techniques to the extracted time series data.
   31. The method according to Aspect 30, wherein extracted time series data comprises three-dimensional coordinates for the vertices of a posture shape at each timepoint.
   32. The method according to Aspect 31, wherein statistical shape analysis is performed on an extracted posture shape.
   33. The method according to Aspect 32, wherein statistical shape analysis is performed on a plurality of extracted posture shapes.
   34. The method according to Aspects 32 or 33, wherein the statistical shape analysis comprises normalizing each posture shape for location, scale, and/or rotational effects.
   35. The method according to Aspect 34, wherein the normalizing comprises determining a mean shape or consensus configuration.
   36. The method according to Aspects 34 or 35, wherein the normalizing comprises performing a generalized Procrustes analysis (GPA).
   37. The method according to any of Aspects 34 to 36, wherein the normalizing comprises transforming the posture shapes into a shape space.
   38. The method according to Aspect 37, wherein the shape space is a Procrustes shape space.
   39. The method according to any of Aspects 32 to 38, wherein the statistical shape analysis comprises reducing the dimensionality and/or degrees of freedom of each posture shape.
   40. The method according to Aspect 39, wherein the dimensionality reduction is performed GPA.
   41. The method according to Aspect 39, wherein the dimensionality reduction is performed using linear methods.
   42. The method according to any of Aspects 39 to 41, wherein the dimensionality reduction is performed using machine learning techniques.
   43. The method according to Aspect 42, wherein the machine learning techniques comprise unsupervised machine learning techniques.
   44. The method according to any of Aspects 32 to 43, wherein processed time series data may be generated for two or more performances of the subject of the one or more goal-directed movements.
   45. The method according to any of Aspects 37 to 44, wherein one or more biomedical outcome metrics are generated using a plurality of posture shapes from each performance of the one or more goal-directed movements.
   46. The method according to any of Aspects 37 to 44, wherein one or more biomedical outcome metrics are generated using a single posture shape from each performance of the one or more goal-directed movements.
   47. The method according to any of Aspects 32 to 46, wherein one or more biomedical outcome metrics are generated using Principal Component Analysis (PCA).
   48. The method according to any of Aspects 37 to 46, wherein one or more biomedical outcome metrics are generated using PCA, wherein the PCA is performed by projecting each posture shape from the shape space into a tangent space.
   49. The method according to Aspect 48, wherein one or more biomedical outcome metrics are generated using a linear combination of the Principal Components (PCs).
   50. The method according to Aspect 49, wherein the linear combination of PCs comprises the two PCs explaining the highest proportion of variance.
   51. The method according to Aspect 45, wherein the one or more goal-directed movements is a task comprising transitioning the body from a first posture to a second posture.
   52. The method according to Aspect 51, wherein one or more biomedical outcome metrics are generated using a characteristic of the subject's posture shape motion or trajectory as the subject transitions from the first posture to the second posture.
   53. The method according to Aspect 52, wherein the characteristic comprises one or more of the path distance, path shape, or path orientation of the posture shape motion from the first posture to the second posture in shape space.
   54. The method according to Aspect 53, wherein the characteristic of posture shape motion is quantified using a statistical test.
   55. The method according to Aspect 54, wherein the characteristic of posture shape motion is quantified using a Mantel test.
   56. The method according to Aspect 51, wherein one or more biomedical outcome metrics are generated using a kinematic deviation index (KDI) quantifying the amount the subject's posture shape motion or trajectory deviates from an ideal trajectory as they transition from the first posture to the second posture.
   57. The method according to Aspect 56, wherein the KDI is generated by projecting the posture shapes into tangent space from shape space.
   58. The method according to Aspect 57, wherein the KDI is generated by calculating the deviation between a straight line through tangent space from the first posture to the second posture and the posture trajectory as the subject transitions from the first posture to the second posture through one or more intermediate postures.
   59. The method according to Aspect 58, wherein the deviation is quantified by measuring the sum of squares of the distances between the straight line and the intermediate postures normalized by the trajectory length from the first posture to the second posture.
   60. The method according to any of Aspects 45 to 59, wherein one or more biomedical outcome metrics are generated using a machine learning model.
   61. The method according to any of the preceding aspects, wherein the biomechanical assessment comprises an interpretation of the one or more biomedical outcome metrics.
   62. The method according to any of the preceding aspects, wherein the biomechanical assessment comprises a predicted health outcome.
   63. The method according to Aspect 62, wherein the predicted health outcome comprises the risk of a future injury.
   64. The method according to Aspect 62, wherein the predicted health outcome comprises the risk of developing a specific disease or condition.
   65. The method according to any of the preceding aspects, wherein the biomechanical assessment comprises the diagnosis of a disease or condition.
   66. The method according to any of the preceding aspects, wherein the biomechanical assessment comprises a determination regarding the severity of one or more mobility disorders.
   67. The method according to any of the preceding aspects, wherein the biomechanical assessment comprises an assessment of the subject's fitness for performing a task.
   68. The method according to any of the preceding aspects, wherein the visual recording is generated at two or more timepoints to generate two or more biomechanical assessments.
   69. The method according to Aspect 68, wherein the two or more timepoints are at least a day apart from each other.
   70. The method according to Aspect 69, wherein the two or more timepoints are at least a month apart from each other.
   71. The method according to any of Aspects 68 to 70, wherein a first timepoint of the two or more timepoints occurs after an injury of the subject.
   72. The method according to any of Aspects 68 to 70, wherein a first timepoint of the two or more timepoints occurs before an injury of the subject.
   73. The method according to Aspect 72, wherein a subsequent timepoint occurs after an injury of the subject.
   74. The method according to any of Aspects 68 to 70, wherein a first timepoint of the two or more timepoints occurs after the subject has received a medical intervention.
   75. The method according to any of Aspects 68 to 70, wherein a first timepoint of the two or more timepoints occurs before the subject has received a medical intervention.
   76. The method according to Aspect 75, wherein a subsequent timepoint occurs after the subject has received a medical intervention.
   77. The method according to any of Aspects 68 to 70, wherein the subject has not received medical intervention.
   78. The method according to any of Aspects 68 to 77, wherein the two or more generated biomechanical assessments are used to determine a level of recovery of the subject after an injury.
   79. The method according to any of Aspects 68 to 76, wherein the two or more generated biomechanical assessments are used to determine a level of recovery of the subject after a surgery.
   80. The method according to any of Aspects 68 to 76, wherein the two or more generated biomechanical assessments are used to determine a level of effectiveness of a medical intervention.
   81. The method according to any of Aspects 68 to 80, wherein the two or more generated biomechanical assessments are used to determine a decline in the mobility of the subject.
   82. The method according to any of the preceding aspects, wherein the subject is a human.
   83. The method according to Aspect 82, wherein the human has a mobility disorder.
   84. The method according to Aspect 83, wherein the mobility disorder is arthritis.
   85. The method according to Aspect 82, wherein the human is 60 years of age or older.
   86. The method according to Aspect 82, wherein the human is younger than 60 years of age.
   87. The method according to Aspect 82, wherein the human has experienced an injury.
   88. The method according to Aspect 87, wherein the injury is a musculoskeletal injury.
   89. The method according to Aspect 88, wherein the injury is an injury of the knee.
   90. The method according to Aspects 88 or 89, wherein the injury has occurred in the last year.
   91. The method according to Aspects 88 or 89, wherein the injury has occurred a year or more in the past.
   92. The method according to Aspect 82, wherein the human regularly performs strength training exercises.
   93. The method according to Aspect 82, wherein the human has received surgery.
   94. The method according to Aspect 93, wherein the surgery occurred on the back, a knee, a hip, an ankle, or a shoulder.
   95. The method according to Aspects 93 or 94, wherein the surgery occurred in the last year.
   96. The method according to Aspects 93 or 94, wherein the surgery occurred a year or more in the past.
   97. The method according to any of the preceding aspects, wherein the biomechanical assessment is produced at least in part using a machine learning model.
   98. The method according to any of the preceding aspects, wherein the biomechanical assessment is saved to a database.
   99. The method according to Aspect 98, wherein the database is used to determine a relationship between health outcomes and one or more biomedical outcome metrics.
   100. The method according to Aspect 98, wherein the database is used to determine a relationship between the mobility disorder severity and one or more biomedical outcome metrics.
   101. The method according to Aspect 98, wherein the database is used to determine a relationship between the fitness of a subject for performing a task and one or more biomedical outcome metrics.
   102. The method according to any of Aspects 99 to 101, wherein the relationship is determined at least in part using a machine learning model.
   103. The method according to any of Aspects 99 to 102, wherein the determined relationship is used to generate subsequent biomechanical assessments.
   104. The method according to any of the preceding aspects, wherein the biomechanical assessment is produced using a computer or smartphone.
   105. The method according to of the preceding aspects, wherein the biomechanical assessment is produced using a computer or smartphone app.
   106. A biomechanical analysis system configured to perform the method according to any of Aspects 1 to 105.
   107. A system for generating a biomechanical assessment for a subject, the system comprising:
a display configured to provide visual information instructing the subject to perform one or more goal-directed movements;
a digital recording device configured to generate a visual recording of the subject performing the one or more goal-directed movements;
a processor configured to receive the visual recording generated by the camera; and
memory operably coupled to the processor wherein the memory comprises instructions stored thereon, which when executed by the processor, cause the processor to extract three-dimensional time series data from the visual recording for a plurality of body landmarks of the subject, process the time series data, generate one or more biomedical outcome metrics from the processed time series data, and produce a biomechanical assessment for the subject from the one or more biomedical outcome metrics.
   108. The system according to Aspect 107, wherein the display is an electronic display device.
   109. The system according to Aspect 108, wherein the electronic display device is the screen of a smartphone or personal computer.
   110. The system according to Aspect 108, wherein the electronic display device comprises an augmented reality device.
   111. The system according to any of Aspects 107 to 110, wherein the digital recording device is configured to generate a sequence of visual images over time.
   112. The system according to Aspect 111, wherein the digital recording device is a webcam or smartphone.
   113. The system according to Aspect 106, wherein the digital recording device is an augmented reality device.
   114. The system according to any of Aspects 111 to 113, wherein the digital recording device is a three-dimensional depth camera.
   115. The system according to any of Aspects 111 to 114, wherein the digital recording device is configured to generate a visual recording at a rate of at least 29 frames per second.

### EXAMPLES

As demonstrated in the above disclosure, the present invention has a wide variety of applications. The following examples are put forth so as to provide those of ordinary skill in the art with a complete disclosure and description of how to use the present invention and are not intended to limit the scope of what the inventors regard as their invention nor are they intended to represent that the experiments below are all or the only experiments performed. Those of skill in the art will readily recognize a variety of noncritical parameters that could be changed or modified to yield essentially similar results. Efforts have been made to ensure accuracy with respect to numbers used (e.g. amounts, distances, calculations, etc.) but some experimental errors and deviations should be accounted for.

### Introduction

Musculoskeletal conditions often impede patient biomechanical function. For example, Osteoarthritis (OA) is a leading cause of disability in the United States. However, despite the relevance of biomechanical function as a marker of disease severity and as a target for therapeutic interventions, clinicians rely on subjective functional assessments with poor test characteristics for biomechanical outcomes because more advanced assessments are impractical in the ambulatory care setting [1-3]. One example of a functional test for the lower extremity is the star excursion balance test (SEBT). The SEBT is an assessment of dynamic postural control during which a subject balances on one leg and maximally reaches in each of eight directions with the contralateral leg without falling or shifting weight to the reaching leg. The conventional SEBT output score is the distance reached in each direction. The SEBT has been validated and utilized in various patient populations to study conditions such as osteoarthritis (OA), patellofemoral pain, ankle instability, ligament reconstructions, lower back pain, and athletic injuries [7-12]. Further, SEBT scores have been shown to have discriminative validity between disease states and to have predictive validity for athletic injuries [13-16].

However, administration of the SEBT is prone to error as all eight scores must be recorded manually, with reported intra-rater reliability ranging from 0.67-0.97 and inter-rater reliability ranging from 0.32-0.96 [17-20]. Recent commercially available markerless motion capture (MMC) cameras have been developed that do not require specialized workspaces and equipment [4-6]. Accordingly, to address these limitations, others have attempted to validate administration of the SEBT using motion capture technology. Kanko et al. used traditional motion capture in a specialized setting to administer SEBT to 37 knee OA patients and observed high correlations with manual measurements [21]. Eltouhky compared traditional motion capture to a MMC system for ten patients during a simplified version of the SEBT and observed excellent agreement and consistency in lower extremity joint angles and reach distances [22]. These approaches have primarily confirmed the accuracy and reproducibility of motion capture in administration of the SEBT, but report only conventional kinematic outcomes (e.g. peak joint angles) and do not demonstrate the clinical utility of more advanced statistical methods (e.g. dimensionality reduction).

In contrast to static postural control which refers to the ability to maintain balance in a specific posture, dynamic postural control reflects the ability to balance over the course of completing a task. The conventional SEBT output metric, reach distances, serve as a proxy for dynamic stance leg stability under the assumption that greater postural control allows for greater reach distances. However, no direct assessment of the trunk or stance leg is recorded in the conventional SEBT and, despite most activities of daily living being inherently dynamic, there is no temporal component since the maximal reach is measured at only one time point during the assessment [23].

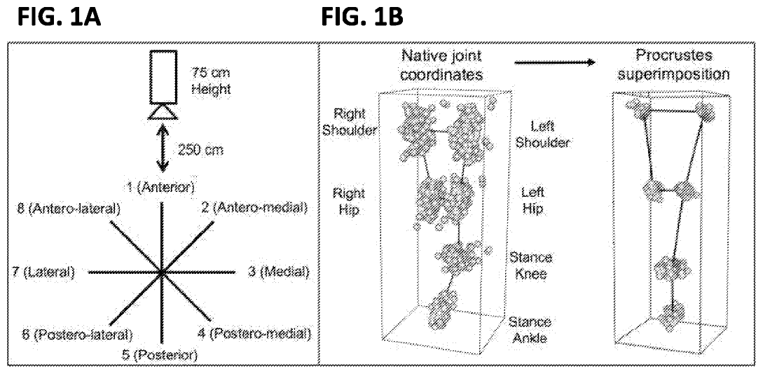

In the following experiments, the accuracy of MMC for SEBT was assessed in the clinical setting. Further, the use of spatiotemporal assessment of the stance leg and trunk during SEBT using MMC for detecting underlying differences in posture kinematics between different disease states beyond conventional SEBT reach distances was explored. Conventional SEBT assessments were performed for three groups of subjects—healthy controls, patients with lower extremity OA, and asymptomatic patients who previously underwent orthopaedic surgical procedures of the lower extremity. Based on the limitations of the conventional SEBT output, MMC was used to quantify and compare movement patterns of the stance leg and trunk using statistical shape modeling (FIG. 1). Finally, a novel kinematic deviation index (KDI) was generated to approximate overall postural control during the assessment and demonstrate both its discriminative ability and its relationship with patient reported health measures in a cohort of OA patients.

FIGS. 1A to 1B depict SEBT reach directions and provide a visual overview of Generalized Procrustes Analysis. (A) an illustration of SEBT configuration, floor grid, and camera orientation. During the assessment, subjects balance on the center of the grid and attempt to reach as far as possible with the other toe in each of the eight noted directions. (B) a visual overview of Generalized Procrustes Analysis with Procrustes superimposition. The raw joint coordinates (right) are transformed with standardization for scaling, rotation, and translation. The resulting superimposed coordinates are on the left.

### Overview of Experiments

A spatiotemporal assessment of patient kinematics was implemented during lower extremity functional testing to evaluate whether kinematic models could identify disease states beyond conventional clinical scoring. 213 trials of the star excursion balance test (SEBT) were recorded by 36 subjects during routine ambulatory clinic visits using both MMC technology and conventional clinician scoring. Conventional clinical scoring failed to distinguish patients with symptomatic lower extremity osteoarthritis (OA) from healthy controls in each component of the assessment. However, principal component analysis of shape models generated from MMC recordings revealed significant differences in subject posture between the OA and control cohorts for six of the eight components. Additionally, time-series models of subject posture change over time revealed distinct movement patterns and reduced overall postural change in the OA cohort compared to the controls. Finally, a novel metric quantifying postural control was derived from subject specific kinematic models and was shown to distinguish OA (1.69), asymptomatic postoperative (1.27), and control (1.23) cohorts (p=0.0025) and to correlate with patient-reported OA symptom severity (R=−0.72, p=0.018). It was found that time series motion data have superior discriminative validity and clinical utility than conventional functional assessments in the case of the SEBT. Based on the experimental findings, novel spatiotemporal assessment approaches were be developed that enable routine in-clinic collection of objective patient-specific biomechanical data for clinical decision-making and monitoring recovery.

### Example 1: Study Population

A total of 213 SEBT trials were performed on 71 legs by 36 subjects during routine ambulatory clinic visits (Table 1). The average subject was 45.7 years old (SD 17.9), with a height of 173.5 cm (SD 10.03) and BMI 27.5 kg/m² (SD 4.25). Of the 36 subjects, 19 were healthy controls, eight had lower extremity OA (three with knee predominant symptoms, and five with hip predominant symptoms), and 9 were asymptomatic postoperative patients undergoing routine follow up. Among the patients in the OA group, the average hip disability and osteoarthritis outcome score (HOOS) and knee injury and osteoarthritis outcome score (KOOS) scores were 37.50 (SD 18.97). There was a significant difference in age between groups (p<0.05), but there was no relationship between groups and sex, height, weight, or BMI.

TABLE 1
Summary of study population.

| | | Age | | Male | | Height | | Weight | | BMI | HOOS/KOOS |
|---|---|---|---|---|---|---|---|---|---|---|---|
| Group | N | (SD) | | (%) | | (SD) | | (SD) | | (SD) | (SD) |
| Controls | 19 | 37.11 | (11.82) | 13 | (68.4) | 174.02 | (9.42) | 82.74 | (14.96) | 27.23 (3.99) | |
| OA | 8 | 70.38 | (9.26) | 5 | (62.5) | 177.96 | (10.05) | 86.50 | (18.26) | 26.93 (3.06) | 37.50 (18.97) |
| Post- | 9 | 42.00 | (14.2) | 5 | (55.5) | 168.43 | (9.02) | 80.69 | (13.28) | 28.61 (5.34) | |

| operative | | | | | | | | | |
|---|---|---|---|---|---|---|---|---|---|
| Totals | 36 | 45.72 | (17.92) | 23 | 173.50 | (10.03) | 83.06 | (15.5) | 27.51 (4.25) |

N, number of participants. SD, standard deviation. OA, lower extremity osteoarthritis. Post-op, asymptomatic post-operative patients. BMI, body mass index. HOOS, Hip disability and osteoarthritis outcome score. KOOS, Knee injury and OA outcome score.

### Example 2: Assessing Accuracy of Conventional SEBT

Repeated measures correlation was employed to compare the relationship between manually measured reach distances and those obtained through MMC. The following are the correlation coefficients for each reach direction: reach one (0.72, 95% CI 0.63-0.79), reach two (0.78, 95% CI 0.7-0.83), reach three (0.64, 95% CI 0.53-0.73), reach four (0.69, 95% CI 0.59-0.77), reach five (0.37, 95% CI 0.22-0.5), reach six (0.34, 95% CI 0.18-0.48), reach seven (0.17, 95% CI −0.01-0.33) and reach eight (0.5, 95% CI 0.36-0.61).

### Example 3: Distinguishing Subject Groups Using Conventional SEBT

In repeated-measures mixed methods linear regression models, leg length-normalized reach distances failed to distinguish OA patients and post-operative patients from controls in any of the eight reach directions (Table 2). Age was modeled as a fixed effect and had a small but statistically significant association with reach distance in direction two (95% CI −0.04-0.00), three (95% CI −0.05-0.01), four (95% CI −0.05-0.00), five (95% CI −0.05-0.00), six (95% CI −0.05-−0.02), seven (9500 CI −0.02-0.00, and eight (95% CI −0.04-0.00). Sex and affected limb status were also modeled as fixed effects but were not associated with reach distances in any direction.

TABLE 2
Repeated-measures mixed methods linear regression models to predict reach distances.

| | | Fixed Effects | | | | | |
|---|---|---|---|---|---|---|---|
| | | Group | | | | | |
| | | | | Post- | | Sex | Affected |
| Direction | | Control | OA | operative | Age | (Male) | Limb |
| 1 | Estimate | Ref | 0.99 | 3.84 | −0.13 | 0.61 | −1.63 |
| | 95% CI | | −0.70-0.90 | −0.31-1.08 | −0.03-0.00 | −0.42-0.54 | −0.82-0.5 |
| | P value | | 0.81 | 0.3 | 0.17 | 0.81 | 0.64 |
| 2 | Estimate | Ref | −0.28 | −1.56 | −0.21 | 2.26 | 2.41 |
| | 95% CI | | −0.86-0.80 | −0.88-0.57 | −0.04-0.00 | −0.27-0.72 | −0.45-0.93 |
| | P value | | 0.95 | 0.68 | 0.03 | 0.39 | 0.51 |
| 3 | Estimate | Ref | 1.05 | −0.7 | −0.31 | 6.72 | 1.97 |
| | 95% CI | | −0.82-1.02 | −0.87-0.73 | −0.05-−0.01 | 0.12-1.22 | −0.56-0.96 |
| | P value | | 0.83 | 0.87 | 0.003* | 0.02 | 0.62 |
| 4 | Estimate | Ref | −8.53 | −1.74 | −0.25 | 5.96 | 2.6 |
| | 95% CI | | −1.81-0.10 | −1.01-0.66 | −0.05-0.00 | 0.03-1.17 | −0.53-1.05 |
| | P value | | 0.1 | 0.07 | 0.02 | 0.05 | 0.53 |
| 5 | Estimate | Ref | −4.88 | 1.01 | −0.27 | 1.71 | 1.12 |
| | 95% CI | | −1.52-0.55 | −0.8-1 | −0.05-0.00 | −0.45-0.79 | −0.75-0.97 |
| | P value | | 0.38 | 0.83 | 0.03 | 0.6 | 0.81 |
| 6 | Estimate | Ref | −4.39 | −3.36 | −0.37 | 0.09 | −0.84 |
| | 95% CI | | −1.22-0.34 | −1.01-0.34 | −0.05-−0.02 | −0.45-0.47 | −0.74-0.57 |
| | P value | | 0.29 | 0.35 | <0.001* | 0.97 | 0.81 |
| 7 | Estimate | Ref | −2.4 | −1.74 | −0.14 | 0.02 | −0.13 |
| | 95% CI | | −0.76-0.27 | −0.6-0.25 | −0.02-0.00 | −0.3-0.3 | −0.46-0.44 |
| | P value | | 0.38 | 0.45 | 0.02 | 0.99 | 0.96 |
| 8 | Estimate | Ref | −3.14 | 0.48 | −0.21 | −3.94 | −0.84 |
| | 95% CI | | −1.08-0.46 | −0.62-0.72 | −0.04-0.00 | −0.85-0.07 | −0.74-0.57 |
| | P value | | 0.44 | 0.89 | 0.02 | 0.11 | 0.81 |

OA, osteoarthritis group. P values < 0.05 are bolded. P values below the Bonferroni corrected threshold of 0.00625 are indicated with an asterisk.

### Example 4: Distinguishing Subject Groups Using Three-Dimensional Posture at Maximal Reach

Three-dimensional coordinates for the stance ankle, stance knee, bilateral hips, and bilateral shoulders were filtered and transformed in a generalized Procrustes analysis (GPA) to normalize body size, translation, and rotation. There was a significant relationship between posture at the time of maximum reach and disease state in six of the eight reach directions after controlling for the effects of age, sex, and affected leg: reach one (p=0.003, F=3.25), reach two (p=0.001, F=6.89), reach three (p=0.001, F=6.4), reach four (p=0.003, F=6.41), reach five (p=0.002, F=5.39), and reach six (p=0.002, F=5.2) (Table 3). No association was observed for directions seven (p=0.35, F=1.09) or eight (p=0.15, F=1.57).

TABLE 3
Relationship between posture at maximal reach and disease state, controlling
for age, sex, and affected leg after Procrustes ANOVA.

| | Group | | | | Age | | | | Sex | | | | Affected Leg | | | |
|---|---|---|---|---|---|---|---|---|---|---|---|---|---|---|---|---|
| D | SS | R² | F | p | SS | R² | F | p | SS | R² | F | p | SS | R² | F | p |
| 1 | 0.05 | 0.09 | 3.25 | 0.003* | 0.004 | 0.01 | 0.46 | 0.752 | 0.01 | 0.02 | 1.27 | 0.278 | 0.002 | 0.003 | 0.23 | 0.913 |
| 2 | 0.14 | 0.16 | 6.89 | 0.001* | 0.02 | 0.03 | 2.32 | 0.069 | 0.04 | 0.05 | 3.91 | 0.023 | 0.001 | 0.001 | 0.09 | 0.991 |
| 3 | 0.17 | 0.14 | 6.4 | 0.001* | 0.05 | 0.04 | 3.81 | 0.018 | 0.1 | 0.08 | 7.06 | 0.007* | 0.001 | 0.001 | 0.07 | 0.993 |
| 4 | 0.22 | 0.14 | 6.41 | 0.003* | 0.11 | 0.07 | 6.31 | 0.01 | 0.12 | 0.08 | 7.28 | 0.003* | 0.008 | 0.005 | 0.48 | 0.621 |
| 5 | 0.24 | 0.13 | 5.39 | 0.002* | 0.16 | 0.08 | 7.08 | 0.004* | 0.06 | 0.03 | 2.62 | 0.069 | 0.005 | 0.002 | 0.2 | 0.886 |
| 6 | 0.22 | 0.12 | 5.2 | 0.002* | 0.25 | 0.14 | 12.24 | 0.001* | 0.09 | 0.05 | 4.25 | 0.017 | 0.006 | 0.003 | 0.31 | 0.827 |
| 7 | 0.04 | 0.03 | 1.09 | 0.35 | 0.11 | 0.08 | 5.66 | 0.003* | 0.03 | 0.02 | 1.42 | 0.22 | 0.007 | 0.005 | 0.35 | 0.856 |
| 8 | 0.06 | 0.04 | 1.57 | 0.15 | 0.01 | 0.01 | 0.5 | 0.71 | 0.1 | 0.07 | 5.41 | 0.002* | 0.015 | 0.011 | 0.82 | 0.491 |

P values < 0.05 are bolded. P values below the Bonferroni corrected threshold of 0.00625 are indicated with an asterisk. D, reach direction. F, F statistic. P, p value. SS, sum of squares.

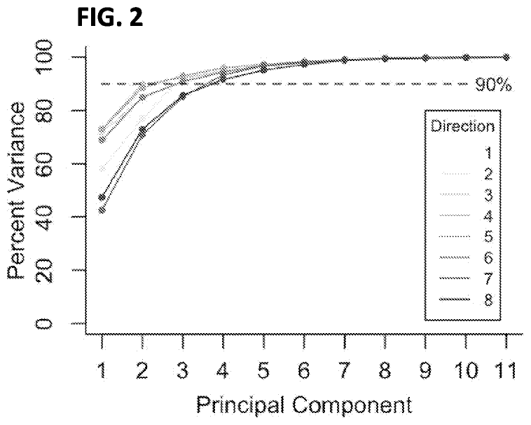

To further investigate these differences in posture following GPA, principal component analyses (PCA) were performed on the posture shape coordinates (i.e., joint centers) in 11-dimensional tangent space, with each principal component (PC) representing a “mode” of posture variation. Four out of the 11 PC vectors accounted for greater than 90% of the overall variance in posture between subjects at the time of maximal reach in each of the eight reach directions (FIG. 2). For each subject, posture at the time of maximal reach was represented as a linear combination of the four PCs explaining the highest proportion of variance in each direction. In an analysis of variance, there were significant relationships between subject group and PC loading in reach one (PC2, p=0.01; PC3, p=0.048), reach two (PC1, p=0.0032; PC2, p=0.0085), reach three (PC1, p=0.0013; PC2, p=0.045), reach four (PC1, p=0.0042; PC2, p=0.0022), reach five (PC1, p=0.0093; PC2, p=0.0021) and reach six (PC1, p=0.0082; PC2, p=0.018). There was no association between subject group and PC loading in reach seven (Table 4).

TABLE 4
Comparison of posture variation between groups at the time of maximal reach.

| | | | | | PC 1: T Test | | PC 2: T Test | |
|---|---|---|---|---|---|---|---|---|
| | ANOVA P Values | | | | vs Controls | | vs Controls | |
| Direction | PC1 | PC2 | PC3 | PC4 | OA | Post-op | OA | Post-op |
| 1 | 0.073 | 0.01 | 0.048 | 0.82 | | | 0.028 | 0.58 |
| 2 | 0.0032* | 0.0085* | 0.14 | 0.25 | <0.001* | 0.94 | 0.011 | 0.17 |
| 3 | 0.0013* | 0.045 | 0.96 | 0.13 | <0.001* | 0.45 | 0.0029* | 0.54 |
| 4 | 0.0042* | 0.0022* | 0.33 | 0.31 | 0.00023* | 0.76 | <0.001* | 0.74 |
| 5 | 0.0093* | 0.0021* | 0.14 | 0.061 | 0.0011* | 0.93 | <0.001* | 0.1 |
| 6 | 0.0082* | 0.018 | 0.87 | 0.93 | 0.0011* | 0.2 | 0.0019* | 0.53 |
| 7 | 0.37 | 0.39 | 0.62 | 0.25 | | | | |
| 8 | 0.097 | 0.9 | 0.14 | 0.79 | | | | |

P values < 0.05 are bolded. P values below the Bonferroni corrected threshold of 0.00625 are indicated with an asterisk. ANOVA, analysis of variance. PC, principal component.

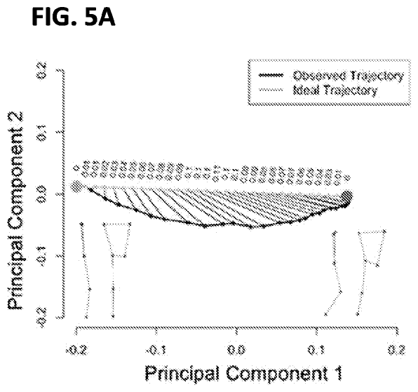

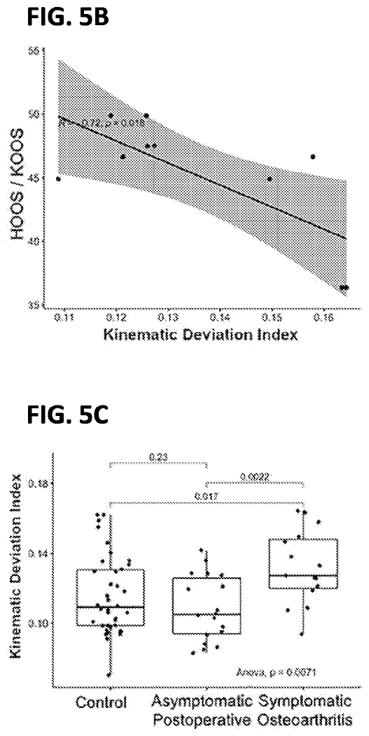

Direct comparisons of the modes of posture variation of OA patients and asymptomatic postoperative patients against the control group were performed using t tests when the ANOVA result was significant. PC loading in the OA cohort was significantly different than the control group in six of the eight reach directions (Table 4). For example, in reach one, patients with OA had significantly lower contributions from PC 2 (35.90% of overall variance) and PC 3 (11.07% of overall variance) than controls (0.028, and 0.037 respectively). The higher contributions from PC 2 among the control group represented greater knee flexion with increased spine extension (FIG. 5). Lower values of PC 3 observed in the OA group were associated with increased knee valgus. In contrast to the OA cohort, no differences were observed between asymptomatic postoperative patients and controls in any reach direction for the two PC's explaining the highest proportion of posture variance (Table 4).

FIG. 2 provides a table illustrating the variance between subjects explained by each principal component for each reach direction. Following principal component analysis, the percentage of overall variance in posture explained by each principal component (i.e., mode of posture variation) was recorded. The first four principal components explained greater than 90% of the variance for each reach direction.

FIGS. 5A to 5C demonstrate the computation of a Kinematic Deviation Index (KDI) and the correlation of computed KDI metrics with patient reported health measures. (A) observed versus ideal trajectories for a representative single subject during a single reach. The black line represents the observed trajectory plotted in principal component space. The grey line represents the theoretical “ideal trajectory” (i.e., straight line through tangent space). The green point represents the initial posture and the red point represents the posture at maximum reach. The actual subject postures are reconstituted in three dimensions for the initial and maximal reach postures. (B) KDI plotted by disease state. Histograms depict raw values for each subject grouped according to cohort. Error bars represent standard error. (C) correlation of KDI with the patient reported health measures, hip disability and osteoarthritis outcome score (HOOS) and knee injury and osteoarthritis outcome score (KOOS).

### Example 5: Distinguishing Subject Groups Based on Time-Series Postural Motion Patterns

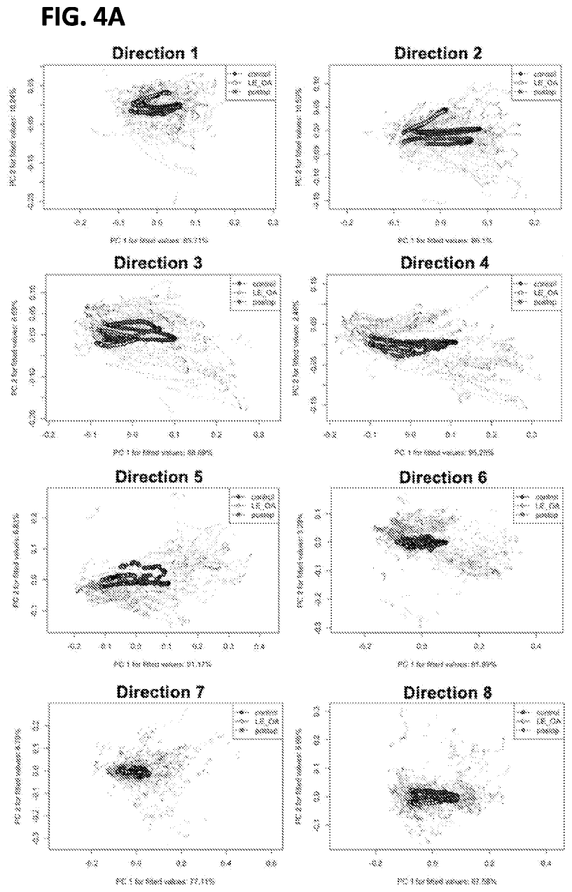

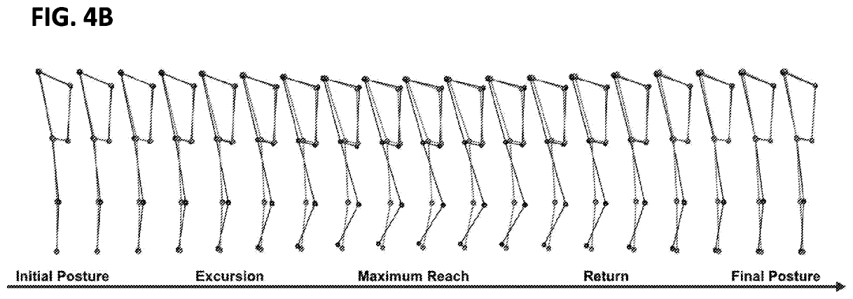

In order to investigate the relationship between time, posture, and disease state, motion during each reach was first represented as ordered sequences of postures through shape space. Mean trajectories for each group, as well as individual subject trajectories, were projected onto the first two PC's explaining the highest proportion of variance and are displayed in FIG. 4. Path distance (total amount of posture change), path shape (how posture changed), and path orientation (the angle between first PC's of posture trajectory) were compared between disease states for each reach direction using Mantel tests due to their high dimensionality. Path distance was significantly shorter in the OA group than the control group in reach two (0.18 vs 0.29, p=0.006), reach three (0.20 vs 0.36, p=0.004), reach four (0.22 vs 0.41, p=0.009), and reach five (0.24 vs 0.40, p=0.037). There were no significant differences in path length between the asymptomatic postoperative patients and the control group.

Compared to control subjects, path shape as measured by the Procrustes distance in shape space was significantly different in the OA patients in reach three (Procrustes distance=0.45, z=2.16, p=0.003) and reach eight (Procrustes distance=0.68, z=1.75, p=0.040). The posture trajectories of asymptomatic postoperative patients were also different than controls in reach two (Procrustes distance=0.46, z=2.04, p=0.015), reach three (Procrustes distance=0.31, z=1.84, p=0.027), and reach five (Procrustes distance=0.59, z=1.92, p=0.023). There were no differences in other reach directions between groups.

Path orientation was significantly different in the OA cohort compared to the controls in reach one (angle=19.4 deg, z=1.73, p=0.036), reach two (angle=40.4 deg, z=4.24, p=0.001), reach three (angle=27.0 deg, z=4.16, p=0.001), and reach four (angle=18.16 deg, z=2.44, p=0.007). Path orientation differed between asymptomatic postoperative patients and controls only in direction three (angle=16.60, z=2.65, p=0.004). There were no significant differences in other reach directions between groups.

FIGS. 4A to 4B illustrate reach trajectories by disease state. (A) reach trajectories are displayed in principal component space for each of the eight reach directions. Each point on the graph represents an entire posture. Each line represents a sequence of postures (i.e., trajectory). Trajectory data for each time point for each subject is plotted in light blue for controls, white for lower extremity osteoarthritis, and red for asymptomatic postoperative patients. The mean trajectory for each group is plotted in dark blue, white, and red for controls, lower extremity osteoarthritis, and for asymptomatic postoperative patients respectively. (B) every third posture along the mean trajectory for the healthy controls (black) and symptomatic osteoarthritis (red) cohorts are plotted in three-dimensional space along the time axis for the second reach direction.

### Example 6: Distinguishing Subject Groups Based on Kinematic Deviation Index and its Relationship with Patient-Reported Health Status

Kinematic deviation index (KDI) was developed as a method to quantify dynamic postural control during the assessment. For each subject, KDI was calculated by comparing the subject's observed posture trajectory in 11-dimensional tangent space to a subject-specific theoretical trajectory with the least overall joint motion (FIG. 5). One KDI score is reported for each patient, which represents KDI averaged over all eight reach distances. In an analysis of variance, there was a significant association between groups and KDI (F=6.56, df=68, ANOVA p=0.0071). In direct comparisons using t tests, patients with OA (mean=1.69, SD=0.49) had significantly greater KDI than both heathy controls (mean=1.23, SD=0.40) (t=−3.19, df=21.52, p=0.0043) and asymptomatic postoperative (mean=1.27, SD=0.41) (t=−2.60, df=27.4, p=0.015) patients.

Within the OA cohort, a significant correlation was observed between KDI and patient-reported Hip Disability and Osteoarthritis Outcome Score and Knee injury and Osteoarthritis Outcome Score scores (R=−0.72, p=0.018).

### SUMMARY

Coupling a single markerless motion capture camera with statistical modeling of posture change, a practical system was developed to perform advanced biomechanical assessments of lower extremity function during routine clinic visits. To validate the system, OA patients and healthy controls were assessed performing a functional balance task by clinicians according to conventional scoring and separately by the motion capture system using kinematic posture modeling. Although clinical scoring failed to distinguish OA patients and healthy controls, the kinematic modeling and dimensionality reduction techniques identified significant differences in both subject posture and motion trajectories throughout the assessment. Furthermore, OA patients reporting more severe symptoms exhibited worse postural control. The results indicate that novel motion capture approaches can enable routine in-clinic collection of objective patient-specific biomechanical data for clinical decision-making and monitoring recovery.

The results of the above experiments demonstrate enhanced clinical utility of time series motion capture data compared to conventional functional tests in the case of the SEBT. First, the accuracy of MMC for recording conventional SEBT reach distances in a clinic setting was validated against manual measurements performed in the standard fashion. Then in a cohort of healthy controls, symptomatic OA patients, and asymptomatic postoperative patients, it was shown that the conventional SEBT reach distances poorly distinguished between groups. However, both time series and static three-dimensional shape models of joint position of the stance leg and trunk did reliably distinguish between groups in most reach directions. Finally, the KDI was proposed to summarize subject performance, which distinguished between disease states and correlated with patient reported outcomes in the cohort of OA patients.

Human motion is the product of complex coordination between the central and peripheral nervous systems and the musculoskeletal system. Because it is currently not possible to observe these interactions directly, existing functional tests rely on proxy endpoint data such as reach distances to assess these underlying systems. In the case of the SEBT, conventional reach distances are a proxy for dynamic postural control. Neuromuscular and musculoskeletal pathologies, as well as subject characteristics impact the body's ability to maintain balance under stress and can affect the observed reach distances during SEBT.

While these proxy endpoints allow for more pragmatic implementation of functional tests in clinic, they are prone to error in their oversimplification of the underlying physiology, and as a result, may miss subtle manifestations of disability. The use of three-dimensional motion trajectory data offers more comprehensive and relevant endpoints. In the case of SEBT, reach distances do not account for the potential impact of alternate reach strategies or compensatory motions to maintain balance. The results indicate that, even in cases where there is no difference in conventional SEBT reach distances between groups, significant differences still persist in three-dimensional posture and time series motion.

A key advantage of MMC with shape modeling over conventional functional tests is the ability to assess relationships between disease state, age, sex, and body characteristics and movement strategies. This has previously been documented in the upper extremity, where sex and age influence movement strategies in subjects reaching towards fixed targets [29]. A prior exploratory factor analysis identified leg length and height as predictors of SEBT reach distances, but found no association between reach distances and sex [30]. Our analysis similarly found no association between sex and reach distance. However, there was a significant relationship between sex and posture at maximal reach in five of the eight reach directions. While males and females may achieve similar reach distances, they may employ different reach strategies based on differences in bone shape, muscle strength, and other parameters. With regards to age, prior studies have suggested younger subjects may reach farther than older subjects [31]. Our study extends prior findings to suggest that while age is not only associated with reach direction, it is also associated with posture at maximal reach. This suggests age-related changes in musculoskeletal physiology and motor control may influence participants to alter their reaching strategies.

Prior attempts have been made to create a composite score for the SEBT, which has generally been described as an average of reach distances [8,32]. Although this score provides a pragmatic method of comparing overall performance between subjects, the reach distance itself is still only a proxy for motor control and does not reflect the results of an underlying biomechanical assessment. The introduction of KDI captures the results of an advanced analysis of three-dimensional motion trajectories in a single numerical score. Subjects performing the SEBT using controlled movements with minimal off target motion travel along a path in shape space more similar to the theoretical ideal motion trajectory. In analysing the experiments, KDI was found to be more discriminative between groups than conventional reach distances, with OA patients demonstrating significantly greater KDI. A recent review found that movement variability during performance of dynamic activities is significantly different in patients with musculoskeletal injury compared to those without, with a trend towards greater movement variability in injured groups [33]. Interestingly, there was no difference in KDI between asymptomatic postoperative patients and healthy controls, suggesting symptom severity may be related to SEBT performance. There was also a correlation between KDI and HOOS and KOOS scores among the OA patients, suggesting that patients who deviate farther from the ideal trajectory during SEBT also subjectively experience worse symptoms.

Current functional tests and movement screens are poor predictors of lower extremity injury risk, and there is a significant need for cohort studies investigating new risk assessment tools [11,34]. As prior studies have shown SEBT performance to be associated with injury risk, KDI may be used as a screening tool [14]. SEBT has also been validated as a tool to track progress in various lower extremity injury patient populations during therapy [8,35,36]. The approach described in this analysis can be practically implemented in most clinics and would provide a quantitative and objective assessment of performance that can be monitored over time. The COVID-19 pandemic has highlighted opportunities for technology to augment existing rehabilitation programs and to offset associated costs, especially in the arthroplasty population [37,38]. Given the low cost and simple configuration of MMC systems, it is feasible for in home and remote deployment.

Strengths of this analysis include a pragmatic application of three-dimensional motion analysis in a clinical setting as well as the introduction of a novel KDI score to capture lower extremity postural control. Further, given that there may be some redundancy in the eight reach directions, a reduction of the number of reaches per trial may occur in order to simplify future data collection. In conclusion, the experiments demonstrate a robust and accessible method for capturing three-dimensional motion data of the lower extremity, demonstrate its utility in distinguishing patient populations, and show the relationship between our analysis and standard patient reported health measures.

### Materials and Methods

### Experimental Design and Population

All experimental protocols and recruitment were approved by the University of California San Francisco Human Research Protection Program. Patients older than 18 years of age were recruited from routine visits to an ambulatory care center for participation in a clinic-based motion analysis session. Informed consent was obtained from each participant. Patients were excluded from the study if any assistive device was required for ambulation or if study personnel determined they were at high risk for a fall based on clinical judgement. Subjects included in the control cohort reported no history of lower extremity pathology requiring treatment (e.g. surgery, nonoperative management). Patients included in the lower extremity OA cohort were receiving treatment for symptomatic hip or knee arthritis, but had no history of joint arthroplasty. Finally, patients with a history of lower extremity orthopaedic surgery who were asymptomatic at the time of the participation were included in a separate group.

### Experimental Configuration and Data Collection

The standard SEBT grid with markings was applied to the floor of a four-by-four meter clinic space with a plain background. During each trial, patients were instructed to reach maximally in each of the eight directions while remaining stable on the stance leg. At the point of maximal reach, patients were instructed to contact the ground with their reaching toe without transitioning weight. Subjects began with the anterior reach direction (direction one), and then proceeded clockwise for right foot reach trials and counter-clockwise for left foot reach trials. Subjects performed two warm up trials on each leg prior to three recorded trials on each leg, and were given unlimited rest between trials. Subjects were instructed to maintain their hands on their hips to minimize use of the arms for balance. If a patient became unstable during a trial, the recording was stopped and the trial was repeated.

A single noninvasive, markerless three-dimensional depth camera (Microsoft Kinect V2, Microsoft, Redmond, WA) was positioned 250 centimeters anterior to the center of the grid at a height of 75 centimeters. The depth camera recorded the positions of the bilateral shoulders, hips, knees, and ankles at a rate of 30 frames per second. Raw joint position data were filtered with a second order low-pass Butterworth filter with a cutoff frequency of three Hz and an allometrically scaled, patient-specific rigid body model [4]. Reach distances were also manually recorded in centimeters as the distance from the stance leg toe to the reach leg toe. To identify the center of the grid using the depth camera, the translation from the center of the grid to the stance ankle were noted during recording, and corresponding adjustments were made during processing based on the averaged location of the stance ankle for each recording. For subjects with lower extremity OA, HOOS or KOOS was recorded [40,41].

### Three-Dimensional Statistical Shape Modeling Procedure

Filtered joint position data for the bilateral shoulders and hips, and stance leg knee and ankle (six total landmarks) for each trial were transformed in a generalized Procrustes analysis (GPA). Since the SEBT is designed to stress the dynamic postural control systems of the stance leg, the reaching leg knee and ankle were not included in this analysis. GPA is the primary method of comparing shape variables from landmark coordinates used in geometric morphometrics [42,43]. In this technique, the three-dimensional joint position data are scaled, rotated, and translated mathematically to minimize the distance between corresponding landmarks between subjects (FIG. 1). Although the dimensionality of the raw subject data is 18 (six landmarks recorded in three dimensions), the aligned Procrustes coordinates following GPA are present in 11-dimensional curved shape space [44]. Seven degrees of freedom are lost during standardization [42]. Data are subsequently projected from curved shape space into Euclidean tangent space for statistical analysis without any additional loss of dimensionality. Data were filtered using MATLAB (The MathWorks Inc, Natick, MA) and GPA was performed in R using the Geomorph (Version 4.0) and RRPP Packages (Version 0.602).

Assessing Accuracy of Conventional SEBT usingWC

All reach distances were normalized to subject leg length, as measured from the anterior superior iliac spine to the medial malleolus, as this has previously been shown to correlate with reach distance [30]. The correlations between manual and depth camera reach distance measurements in each reach direction were assessed using repeated measures correlations. In contrast to the manual measurements which were recorded from the origin to the reach toe, depth camera measurements were recorded from the origin to the reach ankle, due to the higher fidelity of the ankle joint position data compared to the foot marker. The expected offset was confirmed using Bland-Altman plots.

### Distinguishing Subject Groups Using Conventional SEBT

Depth camera reach distances were compared between groups using repeated-measures, mixed methods linear regression models. Repeated measures within subjects (e.g. multiple trials of the same reach direction) were modeled as random intercepts and age, sex, and affected limb status were modeled as fixed effects. P values were estimated using the Satterthwaite's approximation, as this has been shown to produce acceptable type I error. Analysis of reach directions was performed using R (R Foundation, Vienna, Austria).

### Distinguishing Subject Groups Using Three-Dimensional Posture at Maximal Reach

The time of maximal reach was selected for analysis since it corresponds to the conventional SEBT output, reach distance. The first trial for each stance leg per subject was selected for analysis to minimize the potential effect of fatigue on posture. The three-dimensional posture of each subject at maximal reach (i.e., matrix of coordinates of the bilateral shoulders, hips, and stance leg knee and ankle) was recorded after alignment in a generalized Procrustes analysis. To assess the relationship between posture at maximal reach and disease state, Procrustes linear models were generated for each direction of the SEBT controlling for effects of age, sex, and primarily affected leg. Procrustes linear models are fit to the superimposed postures using maximum likelihood estimation on the sum-of-squared Procrustes distances through a residual randomization permutation procedure [45,46].

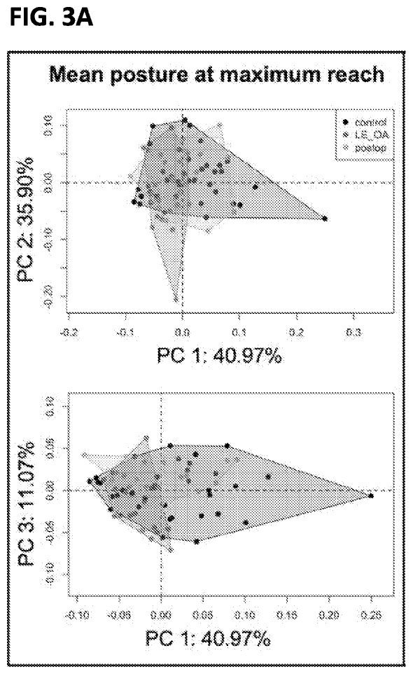

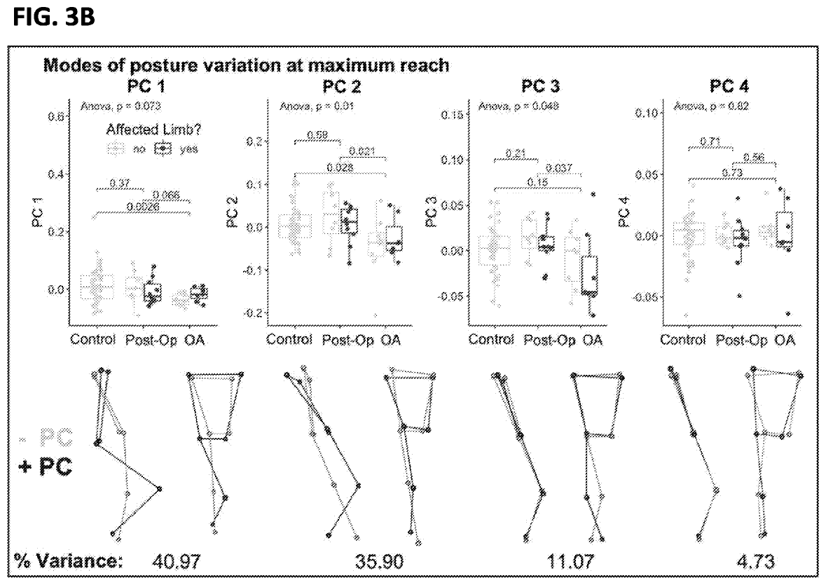

To further investigate between group differences in posture at the time of maximal reach, principal component analysis (PCA) was performed on Procrustes shape coordinates in the tangent plane. Since the Euclidean tangent space is of 11 dimensions, there are 11 principal components for each posture. For each reach direction, the percent of total variance in posture explained by each PC was recorded. Individual subject data were plotted in PC space along the first and second, and first and third principal components containing the highest percentage of variation. Overall group differences in loading for the first four PC's were compared using an ANOVA. The OA and post-operative group loadings were compared directly to controls using T tests if the ANOVA p value was significant. To facilitate interpretation of the PCA results, the minimum and maximum loadings along each of the first four principal components were reconstituted from principal component space to three-dimensional posture space (FIG. 3).

FIGS. 3A to 3B provide the results of principal component analysis of postures at maximal reach. Principal component analysis was performed on maximum reach posture in each of the eight reach directions. Data are presented here for the anterior reach direction (direction one). (A) the posture of each subject at maximum reach is plotted in principal component space along PC1 and PC2 (top) as well as PC1 and PC3 (bottom). Each point on the graph represents a posture. Black circles represent healthy controls. Green circles represent asymptomatic postoperative patients. Red circles represent symptomatic osteoarthritis patients. (B) histograms depict raw principal component values for each subject grouped according to cohort. Error bars represent standard error. The first four modes of posture variation at the time of maximum reach are displayed to visualize the results of the principal component analysis. Dark grey skeletons represent maximum values for that particular mode of variance and light grey skeletons represent the minimum values.

### Distinguishing Subject Groups Based on Time-Series Postural Motion Patterns

SEBT trials were represented as ordered sequences of postures in shape space over time (FIG. 4). Since posture shapes were standardized for size, translation, and rotation in GPA, trajectories represent change in posture during each SEBT reach. As the SEBT was self-paced, motions were defined temporally as the 30 frames prior to and 30 frames following the time of maximal reach in each direction, resulting in 60 frames per trajectory. Three trajectory characteristics were compared between groups: path distance (the extent to which posture changed over each trial), shape (how posture changed during each trial), and orientation (the angle between first principal components of trajectories for each trial). Due to the high dimensionality of the trajectory data (60 observations of six tracked joints in three dimensions), the distance, shape, and orientation of trajectories were compared using Mantel tests [47].

Distinguishing Subject Groups Based on Kinematic Deviation Index and its Relationship with Patient-Reported Health Status

KDI was developed to quantify postural control during the entire SEBT assessment. Mathematically, KDI represents the amount to which a posture trajectory deviates from a theoretical ideal trajectory during a movement. In tangent space, the shortest trajectory from one posture (e.g. rest) to another posture (e.g. maximal reach) is a straight line. Although this path may not represent the path of minimum physiologic energy expenditure, it does represent the theoretical path with the minimum necessary amount of posture change. During motion, deviation from the ideal trajectory occurs when multiple types of posture change occur or when the rate of posture change is variable [48]. To calculate KDI, posture trajectories from resting posture to the point of maximal reach for each reach direction for each subject were identified and transformed using GPA to standardize for shape, translation, and rotation. For each trial, an ideal trajectory was defined as the straight line connecting the rest posture to the point of maximal reach in tangent space. The distances between corresponding time points on the ideal and observed trajectories were calculated for each frame. KDI for each reach was defined as the sum of the squares of these distances normalized by trajectory length (e.g. the total amount of postural change) so as to not penalize subjects undergoing more posture change. Finally, the mean KDI over each of the eight reach directions was reported as the overall KDI for the entire trial.

Regarding interpretation, subjects with higher KDI scores deviated more from the theoretical trajectory (e.g. exhibited multiple types of shape change or temporal variability) and therefore exhibited less postural control. Overall KDI was compared between groups using ANOVA. To assess the relationship between KDI and HOOS and KOOS, Pearson's correlation coefficients were employed.

### REFERENCES

1. Décary S, Ouellet P, Vendittoli P A, Desmeules F. Reliability of physical examination tests for the diagnosis of knee disorders: Evidence from a systematic review. Man Ther. 2016; 26: 172-182. doi:10.1016/j.math.2016.09.007
2. Malanga G A, Andrus S, Nadler S F, McLean J. Physical examination of the knee: A review of the original test description and scientific validity of common orthopedic tests. Arch Phys Med Rehabil. 2003; 84: 592-603. doi:10.1053/apmr.2003.50026
3. Smith T O, Clark A, Neda S, Arendt E A, Post W R, Grelsamer R P, et al. The intra- and inter-observer reliability of the physical examination methods used to assess patients with patellofemoral joint instability. Knee. 2012; 19: 404-410. doi:10.1016/j.knee.2011.06.002
4. Matthew R P, Seko S, Bailey J, Bajcsy R, Lotz J. Estimating Sit-to-Stand Dynamics Using a Single Depth Camera. IEEE J Biomed Heal Informatics. 2019; 23: 2592-2602. doi:10.1109/JBHI.2019.2897245
5. Ngan A, Xiao W, Curran P F, Tseng W J, Hung L W, Nguyen C, et al. Functional workspace and patient-reported outcomes improve after reverse and total shoulder arthroplasty. J Shoulder Elb Surg. 2019; 28: 2121-2127. doi:10.1016/j.jse.2019.03.029
6. Matthew R P, Seko S, Bajcsy R, Lotz J. Kinematic and Kinetic Validation of an Improved Depth Camera Motion Assessment System Using Rigid Bodies. IEEE J Biomed Heal Informatics. 2019; 23: 1784-1793. doi:10.1109/JBHI.2018.2872834
7. Hale S A, Hertel J, Olmsted-Kramer L C. The effect of a 4-week comprehensive rehabilitation program on postural control and lower extremity function in individuals with chronic ankle instability. J Orthop Sports Phys Ther. 2007; 37: 303-311. doi:10.2519/jospt.2007.2322
8. Kanko L E, Birmingham T B, Bryant D M, Gillanders K, Lemmon K, Chan R, et al. The star excursion balance test is a reliable and valid outcome measure for patients with knee osteoarthritis. Osteoarthr Cartil. 2019; 27: 580-585. doi:10.1016/j.joca.2018.11.012
9. Ganesh G S, Chhabra D, Mrityunjay K. Efficacy of the star excursion balance test in detecting reach deficits in subjects with chronic low back pain. Physiother Res Int. 2015; 20: 9-15. doi:10.1002/pri.1589
10. Earl J E, Hertel J. Lower-extremity muscle activation during the star excursion balance tests. J Sport Rehabil. 2001; 10: 93-104. doi:10.1123/jsr.10.2.93
11. Clagg S, Paterno M V., Hewett T E, Schmitt L C. Performance on the modified star excursion balance test at the time of return to sport following anterior cruciate ligament reconstruction. J Orthop Sports Phys Ther. 2015; 45: 444-452. doi:10.2519/jospt.2015.5040
12. Olmsted-Kramer L C, Carcia C, Hertel J, Shultz S. Efficacy of the star excursion balance test in detecting reach deficits in subjects with chronic low back pain. Physiother Res Int. 2015; 20: 9-15. doi:10.1002/pri.1589
13. Al-Khlaifat L, Herrington L C, Tyson S F, Hammond A, Jones R K. The effectiveness of an exercise programme on dynamic balance in patients with medial knee osteoarthritis: A pilot study. Knee. 2016; 23: 849-856. doi:10.1016/j.knee.2016.05.006
14. Plisky P J, Rauh M J, Kaminski T W, Underwood F B. Star excursion balance test as a predictor of lower extremity injury in high school basketball players. J Orthop Sports Phys Ther. 2006; 36: 911-919. doi:10.2519/jospt.2006.2244
15. Gribble P A, Terada M, Beard M Q, Kosik K B, Lepley A S, McCann R S, et al. Prediction of Lateral Ankle Sprains in Football Players Based on Clinical Tests and Body Mass Index. Am J Sports Med. 2016; 44: 460-467. doi:10.1177/0363546515614585
16. McCann R S, Kosik K B, Beard M Q, Terada M, Pietrosimone B G, Gribble P A. Variations in star excursion balance test performance between high school and collegiate football players. J Strength Cond Res. 2015; 29: 2765-2770. doi:10.1519/JSC.0000000000000947
17. Kinzey S J, Armstrong C W. The reliability of the star-excursion test in assessing dynamic balance. J Orthop Sports Phys Ther. 1998; 27: 356-360. doi:10.2519/jospt.1998.27.5.356
18. Lanning C L, Uhl T L, Ingram C L, Mattacola C G, English T, Newsom S. Baseline values of trunk endurance and hip strength in collegiate athletes. J Athl Train. 2006; 41: 427-434.
19. Hertel J, Miller S J, Denegar C R. Intratester and intertester reliability during the star excursion balance tests. J Sport Rehabil. 2000; 9: 104-116. doi: 10.1123/jsr.9.2.104
20. Munro A G, Herrington L C. Between-session reliability of the star excursion balance test. Phys Ther Sport. 2010; 11: 128-132. doi:10.1016/j.ptsp.2010.07.002
21. Kanko L E, Birmingham T B, Bryant D M, Gillanders K, Lemmon K, Chan R, et al. The star excursion balance test is a reliable and valid outcome measure for patients with knee osteoarthritis. Osteoarthr Cartil. 2019; 27: 580-585. doi:10.1016/j.joca.2018.11.012
22. Eltoukhy M, Kuenze C, Oh J, Jacopetti M, Wooten S, Signorile J. Microsoft Kinect can distinguish differences in over-ground gait between older persons with and without Parkinson's disease. Med Eng Phys. 2017; 44: 1-7. doi:10.1016/j.medengphy.2017.03.007
23. Roach K E, Pedoia V, Lee J J, Popovic T, Link™, Majumdar S, et al. Multivariate functional principal component analysis identifies waveform features of gait biomechanics related to early-to-moderate hip osteoarthritis. J Orthop Res. 2021; 39: 1722-1731. doi:10.1002/jor.24901
24. De La Motte S, Arnold B L, Ross S E. Trunk-rotation differences at maximal reach of the Star Excursion Balance Test in participants with chronic ankle instability. J Athl Train. 2015; 50: 358-365. doi:10.4085/1062-6050-49.3.74
25. Robinson R, Gribble P. Kinematic predictors of performance on the star excursion balance test. J Sport Rehabil. 2008; 17: 347-357. doi:10.1123/jsr. 17.4.347
26. Wang L, Tan T, Hu W, Ning H. Automatic gait recognition based on statistical shape analysis. IEEE Trans Image Process. 2003; 12: 1120-1131. doi:10.1109/TIP.2003.815251
27. Matthew R P, Seko S, Bailey J, Bajcsy R, Lotz J. Simple Spline Representation for Identifying Sit-to-Stand Strategies. Proc Annu Int Conf IEEE Eng Med Biol Soc EMBS. 2019; 4097-4103. doi:10.1109/EMBC.2019.8857429
28. Adams D C, Cerney M M. Quantifying biomechanical motion using Procrustes motion analysis. J Biomech. 2007; 40: 437-444. doi:10.1016/j.jbiomech.2005.12.004
29. Chaffin D B, Faraway J J, Zhang X, Woolley C. Stature, age, and gender effects on reach motion postures. Hum Factors. 2000; 42: 408-420. doi:10.1518/001872000779698222
30. Gribble P A, Hertel J. Considerations for normalizing measures of the Star Excursion Balance Test. Meas Phys Educ Exerc Sci. 2003; 7: 89-100. doi:10.1207/S15327841MPEE0702_3
31. Bouillon L E, Baker J L. Dynamic balance differences as measured by the star excursion balance test between adult-aged and middle-aged women. Sports Health. 2011; 3: 466-469. doi:10.1177/1941738111414127
32. Hertel J, Braham R A, Hale S A, Olmsted-Kramer L C. Simplifying the star excursion balance test: Analyses of subjects with and without chronic ankle instability. J Orthop Sports Phys Ther. 2006; 36: 131-137. doi:10.2519/jospt.2006.36.3.131
33. Baida S R, Gore S J, Franklyn-Miller A D, Moran K A. Does the amount of lower extremity movement variability differ between injured and uninjured populations? A systematic review. Scand J Med Sci Sport. 2018; 28: 1320-1338. doi:10.1111/sms.13036
34. Whittaker J L, Booysen N, De La Motte S, Dennett L, Lewis C L, Wilson D, et al. Predicting sport and occupational lower extremity injury risk through movement quality screening: A systematic review. Br J Sports Med. 2017; 51: 580-585. doi:10.1136/bjsports-2016-096760
35. Filipa A, Byrnes R, Paterno M V., Myer G D, Hewett T E. Neuromuscular training improves performance on the star excursion balance test in young female athletes. J Orthop Sports Phys Ther. 2010; 40: 551-558. doi:10.2519/jospt.2010.3325
36. Domingues P C, Serenza F de S, Muniz T B, de Oliveira L F L, Salim R, Fogagnolo F, et al. The relationship between performance on the modified star excursion balance test and the knee muscle strength before and after anterior cruciate ligament reconstruction. Knee. 2018; 25: 588-594. doi:10.1016/j.knee.2018.05.010
37. Bini S, Schilling P, Patel S, Kalore N, Ast M, Maratt J, et al. Digital Orthopedics. A Glimpse Into the Future in the Midst of a Pandemic. Journal of Arthroplasty. 2020. pp. S68-S73. doi:10.1016/j.arth.2020.04.048
38. Bettger J P, Green C L, Holmes D N, Chokshi A, Iii R C M, Hoch B T, et al. Effects of Virtual Exercise Rehabilitation In-Home. J Bone Jt Surg Am. 2020; 0: 101-109.
39. Bell K M, Onyeukwu C, Smith C N, Oh A, Dabbs A D, Piva S R, et al. A portable system for remote rehabilitation following a total knee replacement: A pilot randomized controlled clinical study. Sensors (Switzerland). 2020; 20: 1-16. doi:10.3390/s20216118
40. Klässbo M, Larsson E, Mannevik E. Hip disability and osteoarthritis outcome score: An extension of the Western Ontario and McMaster Universities Osteoarthritis Index. Scand J Rheumatol. 2003; 32: 46-51. doi:10.1080/03009740310000409
41. Roos E M, Roos H P, Lohmander L S, Ekdahl C, Beynnon B D. Knee Injury and Osteoarthritis Outcome Score (KOOS)—Development of a self-administered outcome measure. J Orthop Sports Phys Ther. 1998, 28: 88-96. doi: 10.2519/jospt. 1998.28.2.88
42. Rohlf F J. Shape Statistics: Procrustes Superimpositions and Tangent Spaces. J Classif. 1999; 16: 197-223.
43. Gower J C. Generalized procrustes analysis. Psychometrika. 1975; 40: 33-51. doi:10.1007/BF02291478
44. Kendall D G. Shape manifolds, procrustean metrics, and complex projective spaces. Bull London Math Soc. 1984; 16: 81-121. doi:10.1112/blms/16.2.81
45. Goodall C. Procrustes Methods in the Statistical Analysis of Shape. J R Stat Soc Ser B. 1991; 53: 285-321. doi:10.1111/j.2517-6161.1991.tb01825.x
46. Collyer M L, Sekora D J, Adams D C. A method for analysis of phenotypic change for phenotypes described by high-dimensional data. Heredity (Edinb). 2015; 115: 357-365. doi:10.1038/hdy.2014.75
47. Mantel N. The Detection of Disease Clustering and a Generalized Regression Approach. Cancer Res. 1967; 27: 209-220. doi:10.1136/bmj.3.5668.473-a
48. Martinez C M, McGee M D, Borstein S R, Wainwright P C. Feeding ecology underlies the evolution of cichlid jaw mobility. Evolution (NY). 2018; 72: 1645-1655. doi:10.1111/evo.135181.

In at least some of the previously described embodiments, one or more elements used in an embodiment can interchangeably be used in another embodiment unless such a replacement is not technically feasible. It will be appreciated by those skilled in the art that various other omissions, additions and modifications may be made to the methods and structures described above without departing from the scope of the claimed subject matter. All such modifications and changes are intended to fall within the scope of the subject matter, as defined by the appended claims.

It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (e.g., “a” and/or “an” should be interpreted to mean “at least one” or “one or more”); the same holds true for the use of definite articles used to introduce claim recitations. In addition, even if a specific number of an introduced claim recitation is explicitly recited, those skilled in the art will recognize that such recitation should be interpreted to mean at least the recited number (e.g., the bare recitation of “two recitations,” without other modifiers, means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

In addition, where features or aspects of the disclosure are described in terms of Markush groups, those skilled in the art will recognize that the disclosure is also thereby described in terms of any individual member or subgroup of members of the Markush group. As will be understood by one skilled in the art, for any and all purposes, such as in terms of providing a written description, all ranges disclosed herein also encompass any and all possible sub-ranges and combinations of sub-ranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like include the number recited and refer to ranges which can be subsequently broken down into sub-ranges as discussed above. Finally, as will be understood by one skilled in the art, a range includes each individual member. Thus, for example, a group having 1-3 articles refers to groups having 1, 2, or 3 articles. Similarly, a group having 1-5 articles refers to groups having 1, 2, 3, 4, or 5 articles, and so forth.

Although the foregoing invention has been described in some detail by way of illustration and example for purposes of clarity of understanding, it is readily apparent to those of ordinary skill in the art in light of the teachings of this invention that certain changes and modifications may be made thereto without departing from the spirit or scope of the appended claims. Accordingly, the preceding merely illustrates the principles of the invention. It will be appreciated that those skilled in the art will be able to devise various arrangements which, although not explicitly described or shown herein, embody the principles of the invention and are included within its spirit and scope. Furthermore, all examples and conditional language recited herein are principally intended to aid the reader in understanding the principles of the invention and the concepts contributed by the inventors to furthering the art, and are to be construed as being without limitation to such specifically recited examples and conditions. Moreover, all statements herein reciting principles, aspects, and embodiments of the invention as well as specific examples thereof, are intended to encompass both structural and functional equivalents thereof. Additionally, it is intended that such equivalents include both currently known equivalents and equivalents developed in the future, i.e., any elements developed that perform the same function, regardless of structure. Moreover, nothing disclosed herein is intended to be dedicated to the public regardless of whether such disclosure is explicitly recited in the claims. The scope of the present invention, therefore, is not intended to be limited to the exemplary embodiments shown and described herein. Rather, the scope and spirit of present invention is embodied by the appended claims. In the claims, 35 U.S.C. § 112(f) or 35 U.S.C. § 112(6) is expressly defined as being invoked for a limitation in the claim only when the exact phrase “means for” or the exact phrase “step for” is recited at the beginning of such limitation in the claim; if such exact phrase is not used in a limitation in the claim, then 35 U.S.C. § 112 (f) or 35 U.S.C. § 112(6) is not invoked.

What is claimed is:

1. A method of generating a biomechanical assessment for a subject, the method comprising:
obtaining a visual recording of the subject performing one or more goal-directed movement;
extracting three-dimensional time series data from the visual recording for a plurality of body landmarks of the subject;
processing the time series data;
generating one or more biomedical outcome metrics from the processed time series data; and
producing the biomechanical assessment for the subject from the one or more biomedical outcome metrics.

2. The method according to claim 1, wherein the goal-directed movement comprises a gait movement.

3. The method according to claim 1, wherein the goal-directed movement is performed by the subject in order to complete a task.

4. The method according to claim 3, wherein the task is a functional balance task.

5. The method according to claim 4, wherein the task comprises one or more of the directional reaching tasks of the star excursion balance test (SEBT).

6. The method according to claim 2, wherein the task resembles or is identical to a task associated with the subject's employment.

7. The method according to claim 2, wherein the task is an athletic exercise.

8. The method according to any of claims 3 to 7, wherein the task comprises transitioning the body from a first posture to a second posture.

9. The method according to any of the preceding claims, wherein instructions are provided to the subject guiding the subject through performing the one or more goal-directed movements.

10. The method according to any of the preceding claims, wherein the visual recording is generated without the use of a motion tracking marker.

11. The method according to any of the preceding claims, wherein the visual recording is generated using a three-dimensional depth camera.

12. The method according to any of the preceding claims, wherein the visual recording is generated using a webcam or smartphone.

13. The method according to any of the preceding claims, wherein the visual recording is generated using an augmented reality device.

14. The method according to any of the preceding claims, wherein the visual recording is generated at 29 or more frames per second.

15. The method according to any of the preceding claims, wherein the visual recording is generated at a resolution of 360p or more.

16. The method according to any of the preceding claims, wherein the visual recording is generated at the subject's home.

17. The method according to any of claims 1 to 16, wherein the visual recording is generated at a clinic or hospital.

18. The method according to any of claims 1 to 16, wherein the visual recording is generated at a physical therapy office or studio.

19. The method according to any of the preceding claims, wherein one or more of the plurality of body landmarks comprises a bone or joint of the subject.

20. The method according to claim 19, wherein one or more of the plurality of body landmarks is selected from the group consisting of one or both of the ankles, knees, hips, and shoulders of the subject.

21. The method according to any of the preceding claims, wherein one or more of the plurality of body landmarks is a facial feature of the subject.

22. The method according to any of the preceding claims, wherein the plurality of body landmarks forms a shape characterizing the subject's posture.

23. The method according to any of the preceding claims, wherein the extracted time series data comprises three-dimensional coordinates for the plurality of body landmarks.

24. The method according to claim 23, wherein the processing comprises filtering the extracted time series data.

25. The method according to claim 24, wherein the extracted time series data is filtered using a low pass filter.

26. The method according to claim 25, wherein low pass filter is a Butterworth filter.

27. The method according to any of claims 23 to 26, wherein the time series data is extracted using a machine learning model.

28. The method according to claim 27, wherein the machine learning model comprises a neural network.

29. The method according to claim 28, wherein the neural network is a convolutional neural network.

30. The method according to any of claims 23 to 29, wherein the processing comprises applying kinematic modeling techniques to the extracted time series data.

31. The method according to claim 30, wherein extracted time series data comprises three-dimensional coordinates for the vertices of a posture shape at each timepoint.

32. The method according to claim 31, wherein statistical shape analysis is performed on an extracted posture shape.

33. The method according to claim 32, wherein statistical shape analysis is performed on a plurality of extracted posture shapes.

34. The method according to claim 32 or 33, wherein the statistical shape analysis comprises normalizing each posture shape for location, scale, and/or rotational effects.

35. The method according to claim 34, wherein the normalizing comprises determining a mean shape or consensus configuration.

36. The method according to claim 34 or 35, wherein the normalizing comprises performing a generalized Procrustes analysis (GPA).

37. The method according to any of claims 34 to 36, wherein the normalizing comprises transforming the posture shapes into a shape space.

38. The method according to claim 37, wherein the shape space is a Procrustes shape space.

39. The method according to any of claims 32 to 38, wherein the statistical shape analysis comprises reducing the dimensionality and/or degrees of freedom of each posture shape.

40. The method according to claim 39, wherein the dimensionality reduction is performed GPA.

41. The method according to claim 39, wherein the dimensionality reduction is performed using linear methods.

42. The method according to any of claims 39 to 41, wherein the dimensionality reduction is performed using machine learning techniques.

43. The method according to claim 42, wherein the machine learning techniques comprise unsupervised machine learning techniques.

44. The method according to any of claims 32 to 43, wherein processed time series data may be generated for two or more performances of the subject of the one or more goal-directed movements.

45. The method according to any of claims 37 to 44, wherein one or more biomedical outcome metrics are generated using a plurality of posture shapes from each performance of the one or more goal-directed movements.

46. The method according to any of claims 37 to 44, wherein one or more biomedical outcome metrics are generated using a single posture shape from each performance of the one or more goal-directed movements.

47. The method according to any of claims 32 to 46, wherein one or more biomedical outcome metrics are generated using Principal Component Analysis (PCA).

48. The method according to any of claims 37 to 46, wherein one or more biomedical outcome metrics are generated using PCA, wherein the PCA is performed by projecting each posture shape from the shape space into a tangent space.

49. The method according to claim 48, wherein one or more biomedical outcome metrics are generated using a linear combination of the Principal Components (PCs).

50. The method according to claim 49, wherein the linear combination of PCs comprises the two PCs explaining the highest proportion of variance.

51. The method according to claim 45, wherein the one or more goal-directed movements is a task comprising transitioning the body from a first posture to a second posture.

52. The method according to claim 51, wherein one or more biomedical outcome metrics are generated using a characteristic of the subject's posture shape motion or trajectory as the subject transitions from the first posture to the second posture.

53. The method according to claim 52, wherein the characteristic comprises one or more of the path distance, path shape, or path orientation of the posture shape motion from the first posture to the second posture in shape space.

54. The method according to claim 53, wherein the characteristic of posture shape motion is quantified using a statistical test.

55. The method according to claim 54, wherein the characteristic of posture shape motion is quantified using a Mantel test.

56. The method according to claim 51, wherein one or more biomedical outcome metrics are generated using a kinematic deviation index (KDI) quantifying the amount the subject's posture shape motion or trajectory deviates from an ideal trajectory as they transition from the first posture to the second posture.

57. The method according to claim 56, wherein the KDI is generated by projecting the posture shapes into tangent space from shape space.

58. The method according to claim 57, wherein the KDI is generated by calculating the deviation between a straight line through tangent space from the first posture to the second posture and the posture trajectory as the subject transitions from the first posture to the second posture through one or more intermediate postures.

59. The method according to claim 58, wherein the deviation is quantified by measuring the sum of squares of the distances between the straight line and the intermediate postures normalized by the trajectory length from the first posture to the second posture.

60. The method according to any of claims 45 to 59, wherein one or more biomedical outcome metrics are generated using a machine learning model.

61. The method according to any of the preceding claims, wherein the biomechanical assessment comprises an interpretation of the one or more biomedical outcome metrics.

62. The method according to any of the preceding claims, wherein the biomechanical assessment comprises a predicted health outcome.

63. The method according to claim 62, wherein the predicted health outcome comprises the risk of a future injury.

64. The method according to claim 62, wherein the predicted health outcome comprises the risk of developing a specific disease or condition.

65. The method according to any of the preceding claims, wherein the biomechanical assessment comprises the diagnosis of a disease or condition.

66. The method according to any of the preceding claims, wherein the biomechanical assessment comprises a determination regarding the severity of one or more mobility disorders.

67. The method according to any of the preceding claims, wherein the biomechanical assessment comprises an assessment of the subject's fitness for performing a task.

68. The method according to any of the preceding claims, wherein the visual recording is generated at two or more timepoints to generate two or more biomechanical assessments.

69. The method according to claim 68, wherein the two or more timepoints are at least a day apart from each other.

70. The method according to claim 69, wherein the two or more timepoints are at least a month apart from each other.

71. The method according to any of claims 68 to 70, wherein a first timepoint of the two or more timepoints occurs after an injury of the subject.

72. The method according to any of claims 68 to 70, wherein a first timepoint of the two or more timepoints occurs before an injury of the subject.

73. The method according to claim 72, wherein a subsequent timepoint occurs after an injury of the subject.

74. The method according to any of claims 68 to 70, wherein a first timepoint of the two or more timepoints occurs after the subject has received a medical intervention.

75. The method according to any of claims 68 to 70, wherein a first timepoint of the two or more timepoints occurs before the subject has received a medical intervention.

76. The method according to claim 75, wherein a subsequent timepoint occurs after the subject has received a medical intervention.

77. The method according to any of claims 68 to 70, wherein the subject has not received medical intervention.

78. The method according to any of claims 68 to 77, wherein the two or more generated biomechanical assessments are used to determine a level of recovery of the subject after an injury.

79. The method according to any of claims 68 to 76, wherein the two or more generated biomechanical assessments are used to determine a level of recovery of the subject after a surgery.

80. The method according to any of claims 68 to 76, wherein the two or more generated biomechanical assessments are used to determine a level of effectiveness of a medical intervention.

81. The method according to any of claims 68 to 80, wherein the two or more generated biomechanical assessments are used to determine a decline in the mobility of the subject.

82. The method according to any of the preceding claims, wherein the subject is a human.

83. The method according to claim 82, wherein the human has a mobility disorder.

84. The method according to claim 83, wherein the mobility disorder is arthritis.

85. The method according to claim 82, wherein the human is 60 years of age or older.

86. The method according to claim 82, wherein the human is younger than 60 years of age.

87. The method according to claim 82, wherein the human has experienced an injury.

88. The method according to claim 87, wherein the injury is a musculoskeletal injury.

89. The method according to claim 88, wherein the injury is an injury of the knee.

90. The method according to claim 88 or 89, wherein the injury has occurred in the last year.

91. The method according to claim 88 or 89, wherein the injury has occurred a year or more in the past.

92. The method according to claim 82, wherein the human regularly performs strength training exercises.

93. The method according to claim 82, wherein the human has received surgery.

94. The method according to claim 93, wherein the surgery occurred on the back, a knee, a hip, an ankle, or a shoulder.

95. The method according to claim 93 or 94, wherein the surgery occurred in the last year.

96. The method according to claim 93 or 94, wherein the surgery occurred a year or more in the past.

97. The method according to any of the preceding claims, wherein the biomechanical assessment is produced at least in part using a machine learning model.

98. The method according to any of the preceding claims, wherein the biomechanical assessment is saved to a database.

99. The method according to claim 98, wherein the database is used to determine a relationship between health outcomes and one or more biomedical outcome metrics.

100. The method according to claim 98, wherein the database is used to determine a relationship between the mobility disorder severity and one or more biomedical outcome metrics.

101. The method according to claim 98, wherein the database is used to determine a relationship between the fitness of a subject for performing a task and one or more biomedical outcome metrics.

102. The method according to any of claims 99 to 101, wherein the relationship is determined at least in part using a machine learning model.

103. The method according to any of claims 99 to 102, wherein the determined relationship is used to generate subsequent biomechanical assessments.

104. The method according to any of the preceding claims, wherein the biomechanical assessment is produced using a computer or smartphone.

105. The method according to of the preceding claims, wherein the biomechanical assessment is produced using a computer or smartphone app.

106. A biomechanical analysis system configured to perform the method according to any of claims 1 to 105.

107. A system for generating a biomechanical assessment for a subject, the system comprising:
a display configured to provide visual information instructing the subject to perform one or more goal-directed movements;
a digital recording device configured to generate a visual recording of the subject performing the one or more goal-directed movements;
a processor configured to receive the visual recording generated by the camera; and
memory operably coupled to the processor wherein the memory comprises instructions stored thereon, which when executed by the processor, cause the processor to extract three-dimensional time series data from the visual recording for a plurality of body landmarks of the subject, process the time series data, generate one or more biomedical outcome metrics from the processed time series data, and produce a biomechanical assessment for the subject from the one or more biomedical outcome metrics.

108. The system according to claim 107, wherein the display is an electronic display device.

109. The system according to claim 108, wherein the electronic display device is the screen of a smartphone or personal computer.

110. The system according to claim 108, wherein the electronic display device comprises an augmented reality device.

111. The system according to any of claims 107 to 110, wherein the digital recording device is configured to generate a sequence of visual images over time.

112. The system according to claim 111, wherein the digital recording device is a webcam or smartphone.

113. The system according to claim 106, wherein the digital recording device is an augmented reality device.

114. The system according to any of claims 111 to 113, wherein the digital recording device is a three-dimensional depth camera.

115. The system according to any of claims 111 to 114, wherein the digital recording device is configured to generate a visual recording at a rate of at least 29 frames per second.
